(19) United States
(12) Patent Application Publication
(10) Pub. No.: US 2025/0282886 A1
(43) Pub. Date: Sep. 11, 2025

(54) GLYCAN-MEDIATED PROTEIN DEGRADATION

(71) Applicant: GlycoEra AG, Wädenswil (CH)

(72) Inventors: Amirreza Faridmoayer, Oberengstringen (CH); Rainer Follador, Zurich (CH); Manuela Mally, Watt (CH); Jonathan Albert Back, Villers-le-Lac (CH)

(21) Appl. No.: 18/283,731

(22) PCT Filed: Mar. 22, 2022
(86) PCT No.: PCT/EP2022/057556

§ 371 Date: Sep. 22, 2023

Related U.S. Application Data
(60) Provisional application No. 63/164,963, filed on Mar. 23, 2021.

(51) Int. Cl. C07K 16/30

(52) U.S. Cl. CPC C07K 16/30; C07K 2317/41; C07K 2317/55; C07K 2317/56

(57) ABSTRACT

The present disclosure provides glycoengineered bifunctional binding proteins for glycan-mediated degradation. Such glycan modifications will improve current treatments and allow a better quality of life for patients. Accordingly, such glycoengineered bifunctional binding proteins are useful for treating and preventing various diseases.

### CROSS REFERENCE TO RELATED APPLICATIONS

This application claims the benefit of priority to U.S. Provisional Application No. 63/164,963, filed Mar. 23, 2021, the entire contents of which are incorporated herein by reference.

### FIELD

The present invention relates generally to glycoengineered bifunctional binding proteins having various glycosylation profiles and lysosomal targeting of same.

### BACKGROUND

Endocytic lectins are involved in receptor-mediated endocytosis by capturing glycosylated proteins via specific glycan structures to mediate degradation (Cummings et al., Cold Spring Harbor Laboratory Press, (2017). Endocytic lectins are ubiquitous in humans and can recognize various glycan structures.

Carbohydrate binding receptors are highly diverse and can be exploited by glycoengineering to develop novel therapeutics with unprecedented effectiveness for different diseases, including but not limited to: inflammatory, blood disorders, autoimmune and cancer. This allows development of novel therapeutics based on the concept of glycan-mediated protein degradation. Leveraging natural protein degradation through the glycosylation of monoclonal antibodies can lead to novel therapeutics. To date, it has not been described whether, to what degree, assembly of N-acetylglucosamine (GlcNAc), N-acetyl galactosamine (GalNAc), or galactose (Gal) containing glycans (e.g., GlcNAc2Man3GlcNAc2, GlcNAc1Man3GlcNAc2, GalNAc2GlcNAc2Man3GlcNAc2, GalNAc1GlcNAc2Man3GlcNAc2, GalNAc1GlcNAc1Man3GlcNAc2 or Gal1GlcNAc2Man3GlcNAc2, Gal1GlcNAc1Man3GlcNAc2,Gal2GlcNAc2Man3GlcNAc2) on a protein, can lead to efficient binding by lectins to mediate lysosomal degradation. The present invention shows a novel finding of a specific glycan mediated protein degradation.

The compositions and methods provided herein address the unmet medical need of patients suffering from various difficult to treat diseases such as cancer, autoimmune and inflammatory diseases, and infectious diseases, treated with glycosylated proteins, such as monoclonal antibodies, and provide related advantages.

### SUMMARY

In one aspect, provided herein is a glycoengineered bifunctional binding protein comprising: a first moiety that specifically binds to a target protein associated with a disease; and a second moiety that binds specifically to an endocytic carbohydrate-binding proteins and receptors, wherein the second moiety comprises a glycan structure.

In one aspect, provided herein is a glycoengineered bifunctional binding protein comprising a first moiety that specifically binds to a target protein and a second moiety comprising a glycan comprising terminal GlcNAc.

In one aspect, provided herein is a glycoengineered bifunctional binding protein comprising a first moiety that specifically binds to a target protein and a second moiety comprising a glycan comprising terminal GalNAc.

In one aspect, provided herein is a glycoengineered bifunctional binding protein comprising a first moiety that specifically binds to a target protein and a second moiety comprising a glycan comprising terminal Gal.

In one aspect, provided herein is a glycoengineered bifunctional binding protein comprising a first moiety that specifically binds to a target protein and a second moiety comprising a glycan comprising the structure:

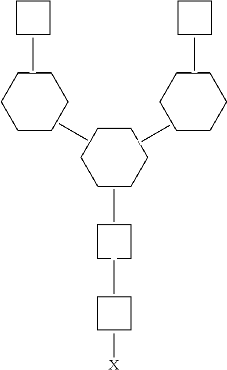

wherein the square represents an N-acetylglucosamine (GlcNAc) residue and the hexagon represents a mannose (Man) residue, and wherein X represents an amino acid residue of the bifunctional binding protein.

In one aspect, provided herein is a glycoengineered bifunctional binding protein comprising a first moiety that specifically binds to a target protein and a second moiety comprising a glycan comprising the structure:

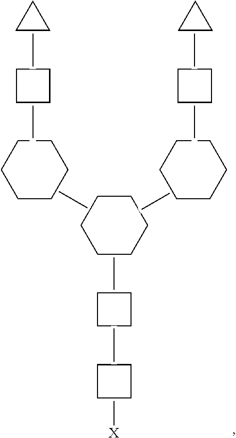

wherein the triangle represents an N-acetyl galactosamine (GalNAc), the white square represents an N-acetylglucosamine (GlcNAc) residue and the hexagon represents a mannose (Man) residue, and wherein X represents an amino acid residue of the bifunctional binding protein.

In one aspect, provided herein is a glycoengineered bifunctional binding protein comprising a first moiety that specifically binds to a target protein and a second moiety comprising a glycan comprising the structure:

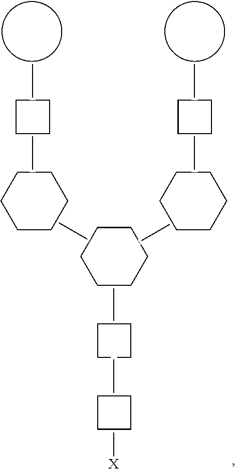

wherein the white circle represents a galactose (Gal) residue, the square represents an N-acetylglucosamine (GlcNAc) residue and the hexagon represents a mannose (Man) residue, and wherein X represents an amino acid residue of the bifunctional binding protein.

In one aspect, provided herein is a glycoengineered bifunctional binding protein, wherein the bifunctional binding protein (i) specifically binds to a target protein and (ii) comprises an N-glycan of the structure:

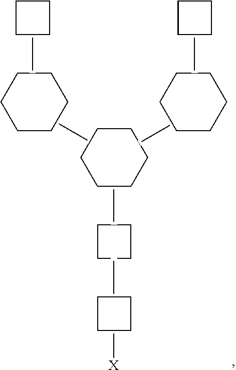

wherein the N-glycan is linked to the bifunctional binding protein at 1, 2, 3, 4 or 5 N-glycosylation sites. In certain embodiments, a bifunctional protein provided herein comprises a N-glycan with a GlcNAc2 as the terminal glycan. Specifically, any branched structure of the N-glycan on the GlcNAc2 part of the N-glycan can also be included.

In one aspect, provided herein is a glycoengineered bifunctional binding protein, wherein the bifunctional binding protein (i) specifically binds to a target protein and (ii) comprises an N-glycan of the structure:

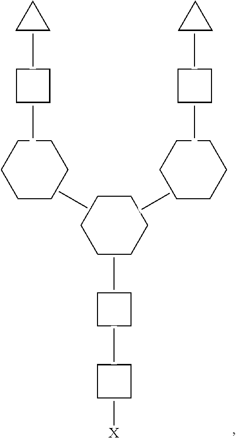

wherein the N-glycan is linked to the bifunctional binding protein at 1, 2, 3, 4 or 5 N-glycosylation sites. In certain embodiments, a bifunctional protein provided herein comprises a N-glycan with a GalNAc2 as the terminal glycan. Specifically, any branched structure of the N-glycan on the GalNAc2 part of the N-glycan can also be included.

In one aspect, provided herein is a glycoengineered bifunctional binding protein, wherein the bifunctional binding protein (i) specifically binds to a target protein and (ii) comprises an N-glycan of the structure:

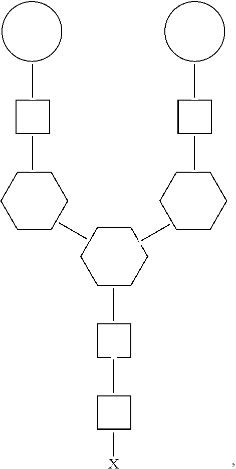

wherein the N-glycan is linked to the bifunctional binding protein at 1, 2, 3, 4 or 5 N-glycosylation sites. In certain embodiments, a bifunctional protein provided herein comprises a N-glycan with a Gal2 as the terminal glycan. Specifically, any branched structure of the N-glycan on the Gal2 part of the N-glycan can also be included.

In certain embodiments, any branched structure of the N-glycan of the proximal GlcNAc (the GlcNAc fused to the glycoengineered bifunctional binding protein) can also be included. In certain embodiments, the proximal GlcNAc can be fucosylated. In certain embodiments, the N-glycan consists of any one of the structures shown above.

In some embodiments, the protein comprises 1 or more, 2 or more, 3 or more, 4 or more, 5 or more, 6 or more, 7 or more, 8 or more, 9 or more or 10 or more glycan structures.

In some embodiments, the glycan structure comprises GlcNAc2Man3GlcNAc2, GalNAc2GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

In some embodiments, the glycan structure is GlcNAc2Man3GlcNAc2.

In some embodiments, the glycan structure is GlcNAc1Man3GlcNAc2

In some embodiments, the glycan structure is GalNAc1GlcNAc2Man3GlcNAc2.

In some embodiments, the glycan structure is GalNAc1GlcNAc1Man3GlcNAc2

In some embodiments, the glycan structure is Gal1 GlcNAc2Man3GlcNAc2

In some embodiments, the glycan structure is Gal1GlcNAc1Man3GlcNAc2.

In some embodiments, the glycan structure is GalNAc2GlcNAc2Man3GlcNAc2.

In some embodiments, the glycan structure is Gal2GlcNAc2Man3GlcNAc2.

In some embodiments, the second moiety specifically binds to a Cluster of Differentiation 206 (CD206) receptor, a DC-SIGN (Cluster of Differentiation 209 or CD209) receptor, a C-Type Lectin Domain Family 4 Member G (LSECTin) receptor, or a macrophage inducible Ca2+-dependent lectin receptor (Mincle).

In some embodiments, the second moiety of the glycoengineered bifunctional binding protein specifically binds to any endocytic carbohydrate-binding receptor recognizing Man3GlcNAc2 structure. In some embodiments, the second moiety of the bifunctional binding protein specifically binds to langerin, a macrophage mannose 2 receptor, dectin-1, dectin-2, BDCA-2, DCIR, MBL, MDL, MICL, CLEC2, DNGR1, CLEC12B, DEC-205, asialoglycoprotein receptor (ASPGR), and mannose 6 phosphate receptor.

In some embodiments, the first moiety of the glycoengineered bifunctional binding protein comprises a heavy chain variable region or a light chain variable region. In some embodiments, the first moiety of the glycoengineered bifunctional binding protein comprises a Fab region of a monoclonal antibody.

In some embodiments, the glycoengineered bifunctional binding protein is an antibody. In some embodiments, the antibody is a monoclonal or polyclonal antibody. In some embodiments, the antibody is recombinant. In some embodiments, the antibody is humanized, chimeric or fully human.

In some embodiments, the antibody has a glycan to protein ratio of 2 to 1, 4 to 1, 6 to 1, 8 to 1, or 10 to 1.

In some embodiments, the glycoengineered bifunctional binding protein is glycosylated at a predetermined and specific residue.

In some embodiments, the glycoengineered bifunctional binding protein is an autoantigen.

In some embodiments, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% of the glycans of the bifunctional binding protein have the same glycan structure.

In some embodiments, the target protein is a cell surface molecule or a non-cell surface molecule. In some embodiments, the cell surface molecule is a receptor. In some embodiments, the non-cell surface molecule is an extracellular protein. In some embodiments, the extracellular protein is an autoantibody, a hormone, a cytokine, a chemokine, a blood protein, or a central nervous system (CNS) protein.

In some embodiments, the target protein is bound by the first moiety.

In some embodiments, the glycan structure comprises Man3GlcNAc, GlcNAc2Man3GlcNAc2, GlcNAc1Man3GlcNAc2, Gal2GlcNAc2Man3GlcNAc2, Gal1GlcNAc2Man3GlcNAc2, GalNAc2GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

In some embodiments, the endocytic carbohydrate-binding proteins and receptors comprise a mannose 3 receptor, mannose binding receptor (CD206), DC-SIGN, L-SIGN, LSECTin, asialoglycoprotein receptor (ASGPR), scavenger receptor C-type lectin (SRCL), mannose-6-phosphate receptor, mincle, dectin-1, dectin-2, langerin, cation-independent mannose 6-phosphate receptor (CI-M6PR), macrophage mannose receptor 2, BDCA-2, human macrophage galactose lectin (MGL), c-type lectin domain family 5, member A (CLEC5A or MDL), myeloid inhibitory c-type lectin-like receptor (MICL), c-type lectin-like receptor 2 (CLEC2), dendritic cell natural killer lectin group receptor 1 (DNGR1), or c-type lectin domain family 12, member B (CLEC12B).

In some embodiments, the disease comprises a cancer or is involved in cancer progression. In some embodiments, the disease comprises an autoimmune disease. In some embodiments, the disease comprises amyloidosis. In some embodiments, the amyloidosis can be systemic amyloidosis. In some embodiments, the amyloidosis can be localized amyloidosis.

In some embodiments, the target protein associated with a disease is upregulated in the disease compared to a non-disease state. In some embodiments, the target protein associated with a disease is expressed in the disease compared to a non-disease state.

In some embodiments, the target protein associated with said disease comprises TNFα, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPα, CD38, CD73, TGF-β, Bombesin R, CAIX, CD13, CD44v6, Emmprin, Endoglin, EpCAM, FAP-α, Folate R, GRP78, IGF-1R, Matriptase, Mesothelin, sMET/HGFR, MT1-MMP, MT6-MMP, PSCA, PSMA, Tn antigen, and uPAR, TSHRα, Myelin oligodendrocyte glycoprotein (MOG), AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, LILRB1, LILRB2, VEGF-R, CXCR4, CXCL12, CSF-1, CD47, aggregated light chain or aggregated transthyretin.

In some embodiments, the target protein associated with said autoimmune disease is an antibody binding to TSHRα, MOG, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin, or gangliosides GM1, GD3 or GQ1B.

In some embodiments, the target protein is upregulated or expressed in tumor associated macrophages (TAMs). In some embodiments, the target protein is associated with TAMs recruitment in the tumor microenvironment. In other embodiments, the target protein is associated with TAMs depletion in the tumor microenvironment. In further embodiments, the target protein is associated with TAMs reprogramming in the tumor microenvironment.

In some embodiments, the target protein associated with TAMs comprises SIRPα, CCR2, CSF-1R, LILRB1, LILRB2, VEGF-R, or CXCR4. In other embodiments, the target protein associated with TAMs comprises CCL2, CXCL12, CSF-1 or CD47.

In some embodiments, the endocytic carbohydrate-binding proteins and receptors comprise a DC-SIGN, L-SIGN, LSECTin, asialoglycoprotein receptor (ASGPR), mannose-6-phosphate receptor, mincle, dectin-1, dectin-2, langerin, cation-independent mannose 6-phosphate receptor (CI-MPR), macrophage mannose receptor 2, BDCA-2, MGL, MDL, MICL, CLEC2, DNGR1, or CLEC12B.

In some embodiments, the disease comprises a cancer.

In some embodiments, the disease comprises an autoimmune disease.

In one aspect, provided herein is a method of delivering a target protein to a hepatocyte endosome comprising: contacting the target protein with the glycoengineered bifunctional binding protein provided herein under conditions to mediate endocytosis of the target protein.

In some embodiments, the number of glycan structures on the protein increases the rate of delivery.

In some embodiments, the number of glycan structures comprise 1 or more, 2 or more, 3 or more, 4 or more, 5 or more, 6 or more, 7 or more, 8 or more, 9 or more or 10 or more.

In some embodiments, the glycan structure comprises GlcNAc2Man3GlcNAc2, GalNAc2GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

In some embodiments, the glycan structure is GlcNAc2Man3GlcNAc2.

In some embodiments, the glycan structure is GalNAc2GlcNAc2Man3GlcNAc2.

In some embodiments, the glycan structure is Gal2GlcNAc2Man3GlcNAc2.

In one aspect, provided herein is a method of degrading a target protein comprising: contacting the target protein with the glycoengineered bifunctional binding protein provided herein under conditions to mediate lysosomal degradation of the target protein by a host cell.

In some embodiments, the number of glycan structures on the protein increases the rate of lysosomal degradation.

In some embodiments, the number of glycan structures comprise 1 or more, 2 or more, 3 or more, 4 or more, 5 or more, 6 or more, 7 or more, 8 or more, 9 or more or 10 or more.

In some embodiments, the glycan structure comprises GlcNAc2Man3GlcNAc2, GalNAc2GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

In some embodiments, the glycan structure is GlcNAc2Man3GlcNAc2.

In some embodiments, the glycan structure is GalNAc2GlcNAc2Man3GlcNAc2.

In some embodiments, the glycan structure is Gal2GlcNAc2Man3GlcNAc2.

In some embodiments, the target protein is upregulated in cancer or involved in cancer progression. In some embodiments, the target protein upregulated in cancer or involved in cancer progression comprises HER2, EGFR, HER3, VEGFR CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPα, CD38, CD73, or TGF-β.

In some embodiments, the target protein is an autoantibody of an autoimmune disease. In some embodiments, the target protein is an autoantigen in an autoimmune disease.

In some embodiments, the autoantibody in the autoimmune disease is an antibody binding to TSHRα, Myelin oligodendrocyte protein (MOG), AChR-α1, noncollagen domain 1 of the α3 chain of type IV collagen (α3NC1), ADAMTS13, Desmoglein-1/3, GPIb/IX, GPIIb/IIIa, GPIa/IIa, NMDA receptor, glutamic acid decarboxylase (GAD), amphiphysin, or gangliosides GM1, GD3 or GQ1B.

In some embodiments, the target protein is upregulated or expressed in a neurodegenerative disease. In some embodiments, the target protein upregulated or expressed in a neurodegenerative disease is alpha-synuclein, amyloid beta or complement cascade component.

In some embodiments, the target protein is upregulated in amyloidosis. In some embodiments, the amyloidosis can be systemic amyloidosis. In other embodiments, the amyloidosis can be localized amyloidosis. In some embodiments, the protein upregulated in systemic amyloidosis can be transthyretin.

In some embodiments, the host cell is a liver cell, myeloid cell, an immune cell, an endothelial cell, a parenchymal cell or an epithelial cell. In some embodiments, the immune cell is a dendritic cell, a macrophage, a monocyte, a microglia cell, a granulocyte or a B lymphocyte.

In some embodiments, the host cell is any cell.

In some embodiments, the glycoengineered bifunctional binding protein enhances degradation of the target protein relative to degradation of the target protein in the presence of a glycoengineered bifunctional binding protein comprising a different second moiety.

In some embodiments, degradation is mediated by endocytosis or phagocytosis.

In one aspect, provided herein is a pharmaceutical composition comprising the glycoengineered bifunctional binding protein provided herein and a pharmaceutically acceptable carrier.

In one aspect, provided herein is a method of treating or preventing a disease in a patient comprising: administering to the patient the glycoengineered bifunctional binding protein provided herein, or the pharmaceutical composition provided herein.

In some embodiments, the disease is an autoimmune disease, a cancer or tumor, a liver disease, an inflammatory disorder, or a blood coagulation disorder.

In some embodiments, the autoimmune disease is selected from Graves' Disease, Myelin oligodendrocyte glycoprotein antibody-associated disease (MOGAD), Myasthenia Gravis, Anti-GBM Disease, Immune Thrombotic Thrombocytopenia Purpura, Acquired Pemphigus Vulgaris, Immune Thrombocytopenia, autoimmune encephalitis, Guillain-Barré Syndrome, and Membranous Nephropathy.

In some embodiments, the cancer comprises a solid tumor. In some embodiments, the cancer comprises a blood-borne cancer or tumor. In some embodiments, the cancer may be a carcinoma or a sarcoma. In some embodiments, the cancer is selected from lung cancer (small cell or non-small cell), breast cancer, gastric cancer, colorectal cancer, bladder cancer, malignant melanoma, brain cancer (e.g., astrocytoma, glioma, meningioma, neuroblastoma, or others), bone cancer (e.g., osteosarcoma), cervical cancer, cholangiocarcinoma, digestive tract cancer (e.g., oral, esophageal, stomach, colon or rectal cancer), head and neck cancer, leiomyosarcoma, liposarcoma, liver cancer (e.g., hepatocellular carcinoma), mesothelioma, nasopharyngeal cancer, neuroendocrine cancer, ovarian cancer, pancreatic cancer, prostate cancer, renal cancer, rhabdomyosarcoma, salivary gland cancer, skin cancer, spindle cell carcinoma, testicular cancer, thyroid cancer, or uterine cancer (e.g., endometrial cancer). In certain embodiments, the cancer can be relapsed following a previous therapy, or refractory to conventional therapy. In certain embodiments, the cancer can be disseminated or metastatic. In some embodiments, the blood-borne cancer or tumor is selected from leukemia, myeloma (e.g., multiple myeloma) lymphoma (e.g., Hodgkin's lymphoma or non-Hodgkin's lymphoma). In certain embodiments, the leukemia is chronic lymphocytic leukemia, chronic myeloid leukemia, acute lymphocytic leukemia, acute myelogenous leukemia and acute myeloblastic leukemia.

In some embodiments, treatment comprises reprogramming tumor associated macrophages (TAMs) by administering the bifunctional binding protein under conditions to mediate endocytosis of a target protein.

In some embodiments, the target protein is upregulated or expressed in TAMs.

In some embodiments, the target protein upregulated or expressed in TAMs comprises SIRPα, CCR2, CSF-1R, LILRB1, LILRB2, VEGF-R, CXCR4, CCL2, CXCL12, CSF-1 or CD47.

In some embodiments, the administration step comprises intravenous injection, intraperitoneal injection, subcutaneous injection, transdermal injection, or intramuscular injection.

In one aspect, provided herein is a kit comprising the glycoengineered bifunctional binding protein provided herein, or the pharmaceutical composition provided herein and instructions for administering the glycoengineered bifunctional molecule or pharmaceutical composition to an individual in need thereof.

In some embodiments, the glycoengineered bifunctional binding protein or pharmaceutical composition is present in one or more unit dosages.

In one aspect, provided herein is a method of degrading a target protein in a subject comprising administering a bifunctional binding protein, wherein the bifunctional binding protein specifically binds to the target protein and comprises a biantennary GalNAc capable of binding asialoglycoprotein receptor (ASGPR).

In some embodiments, the bifunctional binding protein comprises biantennary GalNAc.

In some embodiments, the biantennary GalNAc has the following structure:

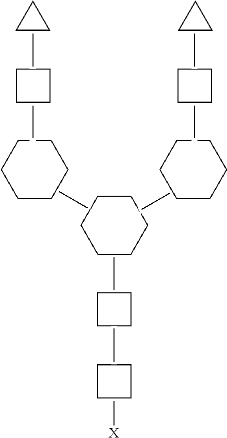

wherein the triangle represents N-acetylgalactosamine (GalNAc), the white square represents N-acetylglucosamine (GlcNAc), the hexagon represents mannose (Man), and the X represents an amino acid residue.

In one aspect, provided herein is a method of treating an acute condition associated with increased levels of a target protein, wherein the method comprises administering to a patient in need thereof a bifunctional binding protein, wherein the bifunctional binding protein (i) specifically binds to the target protein and (ii) comprises an N-glycan, wherein the N-glycan is linked to the bifunctional binding protein at one, two or more glycosites such that the half-life of the target protein in the patient is at most 15 minutes, 30 minutes, 45 minutes, 60 minutes, 90 minutes, or at most 2 hours.

In one aspect, provided herein is a method of treating an acute condition associated with increased levels of a target protein, wherein the method comprises administering to a patient in need thereof a bifunctional binding protein, wherein the bifunctional binding protein (i) specifically binds to the target protein and (ii) comprises an N-glycan, wherein the N-glycan is linked to the bifunctional binding protein at one, two or more glycosites such that the half-life is at most 20%, 30%, 40%, 50%, 60%, 70%, 80%, 90% or 99% of the half-life of the target protein in the patient in the absence of the bifunctional binding protein or in the absence of any treatment.

In some embodiments, the N-glycan binds asialoglycoprotein receptor (ASGPR) and the N-glycan has the structure of:

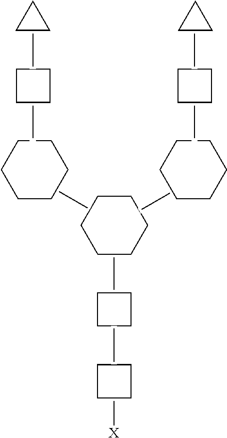

wherein the triangle represents N-acetylgalactosamine (GalNAc), the white square represents N-acetylglucosamine (GlcNAc), the hexagon represents mannose (Man), and the X represents an amino acid residue.

In some embodiments, the bifunctional binding protein comprises GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

In one aspect, provided herein is a method of treating a chronic condition associated with increased levels of a target protein, wherein the method comprises administering to a patient in need thereof a bifunctional binding protein, wherein the bifunctional binding protein (i) specifically binds to the target protein and (ii) comprises an N-glycan wherein the N-glycan is linked to the bifunctional binding protein at most one or more glycosites, such that the half-life of the target protein in the patient is at least 12 hours, 1 day, 2 days or 3 days.

In one aspect, provided herein is a method of treating a chronic condition associated with increased levels of a target protein, wherein the method comprises administering to a patient in need thereof a bifunctional binding protein, wherein the bifunctional binding protein (i) specifically binds to the target protein and (ii) comprises an N-glycan, wherein the N-glycan is linked to the bifunctional binding protein at one or more glycosites, such that the half-life is at most 20%, 30%, 40%, 50%, 60%, 70%, 80%, 90% or 99% of the half-life of the target protein in the patient in the absence of the bifunctional binding protein or in the absence of any treatment.

In some embodiments, the N-glycan binds asialoglycoprotein receptor (ASGPR) and wherein the N-glycan is biantennary GalNAc having the structure:

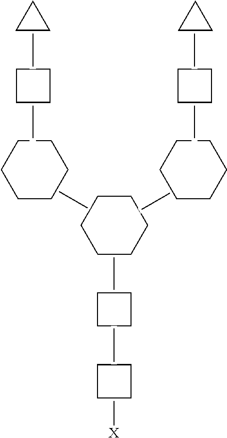

wherein the triangle represents N-acetylgalactosamine (GalNAc), the white square represents N-acetylglucosamine (GlcNAc), the hexagon represents mannose (Man), and the X represents an amino acid residue.

In some embodiments, the bifunctional binding protein comprises GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

In some embodiments, the chronic condition is an autoimmune disease, a cancer or tumor, a liver disease, an inflammatory disorder, or a blood coagulation disorder.

In some embodiments, the autoimmune disease is selected from Graves' Disease, Myasthenia Gravis, Anti-GBM Disease, Immune Thrombotic Thrombocytopeniaurpura, Acquired Pemphigus Vulgaris, Immune Thrombocytopenia, autoimmune encephalitis, Guillain-Barré Syndrome, and Membranous Nephropathy.

In some embodiments, the cancer comprises a solid tumor. In some embodiments, the cancer comprises a blood-borne cancer or tumor. In some embodiments, the cancer may be a carcinoma or a sarcoma. In some embodiments, the cancer is selected from lung cancer (small cell or non-small cell), breast cancer, gastric cancer, colorectal cancer, bladder cancer, malignant melanoma, brain cancer (e.g., astrocytoma, glioma, meningioma, neuroblastoma, or others), bone cancer (e.g., osteosarcoma), cervical cancer, cholangiocarcinoma, digestive tract cancer (e.g., oral, esophageal, stomach, colon or rectal cancer), head and neck cancer, leiomyosarcoma, liposarcoma, liver cancer (e.g., hepatocellular carcinoma), mesothelioma, nasopharyngeal cancer, neuroendocrine cancer, ovarian cancer, pancreatic cancer, prostate cancer, renal cancer, rhabdomyosarcoma, salivary gland cancer, skin cancer, spindle cell carcinoma, testicular cancer, thyroid cancer, or uterine cancer (e.g., endometrial cancer). In certain embodiments, the cancer can be relapsed following a previous therapy, or refractory to conventional therapy. In certain embodiments, the cancer can be disseminated or metastatic. In some embodiments, the blood-borne cancer or tumor is selected from leukemia, myeloma (e.g., multiple myeloma) lymphoma (e.g., Hodgkin's lymphoma or non-Hodgkin's lymphoma). In certain embodiments, the leukemia is chronic lymphocytic leukemia, chronic myeloid leukemia, acute lymphocytic leukemia, acute myelogenous leukemia and acute myeloblastic leukemia. In some embodiments, treatment comprises reprogramming tumor associated macrophages (TAMs) by administering the bifunctional binding protein under conditions to mediate endocytosis of a target protein.

In some embodiments, the target protein is upregulated or expressed in TAMs.

In some embodiments, the target protein upregulated or expressed in TAMs comprises SIRPα, CCR2, CSF-1R, LILRB1, LILRB2, VEGF-R, CXCR4, CCL2, CXCL12, CSF-1 or CD47.

In some embodiments, the administration step comprises intravenous injection, intraperitoneal injection, subcutaneous injection, transdermal injection, or intramuscular injection.

### BRIEF DESCRIPTION OF THE DRAWINGS

FIG. 1 shows examples of glycan mediated modes of action that can be used for therapy, including glycan mediated degradation.

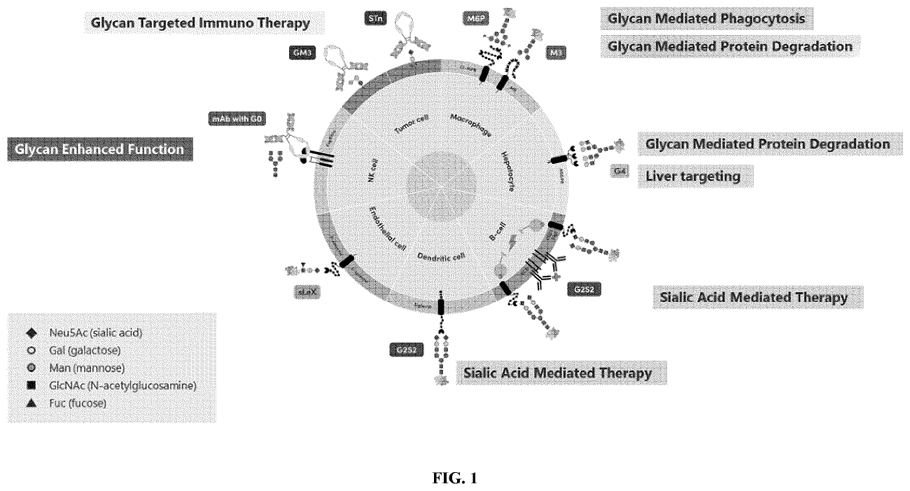

FIG. 2 shows asialoglycoprotein receptor (ASGPR) mediated degradation of extracellular proteins in the hepatocyte.

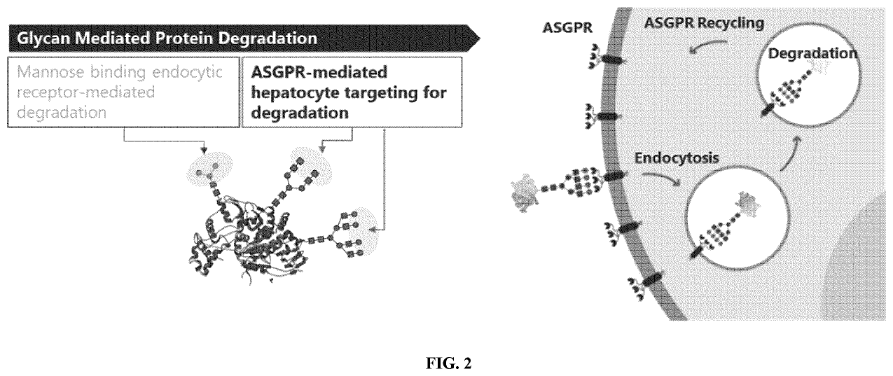

FIG. 3 shows glycan mediated protein degradation via asialoglycoprotein receptor (ASGPR).

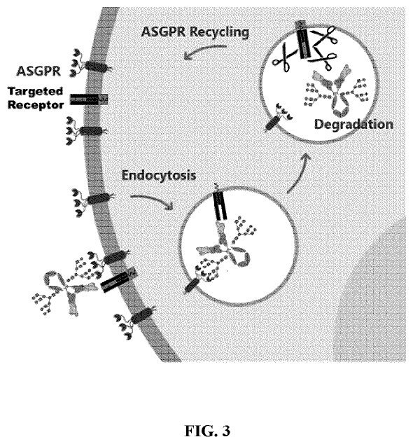

FIG. 4 shows that an antibody displaying A2GalNAc2 glycan on Fab is highly internalized by HepG2 cells. HepG2 cells were incubated for 4 hours with pHrodo-labeled antibodies. The graph shows the average adjusted mean fluorescence intensities (MFI) of pHrodo of triplicate values±standard error of the mean (SEM). Graph shows data from one out of 3 representative experiments.

FIG. 5 shows that internalization of antibodies displaying A2GalNAc2 glycan on Fab by HepG2 cells is mediated by ASGPR. HepG2 cells were incubated for 3 hours with pHrodo-labeled antibodies (3 μg/ml) and indicated inhibitors. The graph shows the average and individual adjusted pHrodo MFI of 2 independent experiments. Black circle: No inhibitor. Black triangle: Fetuin. Open square: Asialofetuin. Black diamond: EGTA. Open circle: Chloroquine. Open triangle: Bafilomycin.

FIG. 6 shows that antibodies displaying A2 Glycan structure lead to potent elimination of a target antigen from blood circulation in rat, depending on their number of Fab glycan displayed. Rats were injected intravenously (i.v) with HCA202 (0.5 mg/kg) and with Antibodies i.v 10 mg/kg. Graph shows average±standard deviation (SD) of HCA202 serum concentration in ng/ml of 3 or 4 animal/group. Black circles show H-A2F (adalimumab, Humira) treated group. Open squares show A-84-A2 treated group. Black triangles show A-8486-A2 treated group. Black square show A-8486-A2G2S2 treated group. Open circles show PBS treated group (HCA202 only). Open diamonds and dotted line show A-M3 treated group. For graphical representation, when HCA202 levels were below assay Lower Limit of Quantification (LLOQ) at the minimal required 10 fold dilution (MRD10) (dotted LLOQ/MRD10 line=20 ng/ml), they were set at 19 ng/ml.

FIG. 7 shows that antibodies displaying A2G2 Glycan structure lead to potent elimination of a target antigen from blood circulation in rat, depending on their number of Fab glycans displayed. Rats were injected i.v with HCA202 (0.5 mg/kg) and with Antibodies i.v 10 mg/kg. Graph shows average±SD of HCA202 serum concentration in ng/ml of 3 or 4 animal/group. Black circles show H-A2F (adalimumab, Humira) treated group. Open squares show A-84-A2G2 treated group. Black triangles show A-8486-A2G2 treated group. Black square show A-8486-A2G2S2 treated group. Open circles show PBS treated group (HCA202 only). Open diamonds and dotted line show A-M3 treated group. For graphical representation, when HCA202 levels were below assay LLOQ (dotted LLOQ/MRD10 line=20 ng/ml), they were set at 19 ng/ml.

FIG. 8 shows that antibodies displaying A2GalNAc2 Glycan structure lead to potent elimination of a target antigen from blood circulation in rat. Rats were injected i.v with HCA202 (0.5 mg/kg) and with Antibodies i.v 10 mg/kg. Graph shows average±SD of HCA202 serum concentration in ng/ml of 3 or 4 animal/group. Black circles show H-A2F (adalimumab, Humira) treated group. Open squares show A-84-A2GalNAc2 treated group. Black triangles show A-8486-A2GalNAc2 treated group. Black square show A-8486-A2G2S2 treated group. Open circles show PBS treated group (HCA202 only). Open diamonds and dotted line show A-M3 treated group. For graphical representation, when HCA202 levels were below assay LLOQ (dotted LLOQ/MRD10 line=20 ng/ml), they were set at 19 ng/ml.

FIG. 9 shows that an antibody displaying A2G2 Glycan structure on Fab is distributed to the liver area as compared to control antibodies. Mice were injected i.v with CF750-labeled antibodies at 5 mg/kg and imaged using fluorescence tomography. The graph shows the average fluorescence in pmol±SD of 3 animals/time point in the gated liver region of interest. Open Squares and dotted line show Ptz-A2F treated group. Black Circles show H-A2F treated group. Open diamonds show A-84865-A2G2S2 treated group. Black triangles show A-8486-A2G2 treated group.

FIG. 10 shows that an antibody displaying A2GalNAc2 Glycan structure is distributed to the liver area with a fast kinetic as compared to control antibodies. Mice were injected i.v with CF750-labeled antibodies at 5 mg/kg and imaged using fluorescence tomography. The graph shows the average fluorescence in pmol±SD of 3 animals/time point in the gated liver region of interest. Open Squares and dotted line show Ptz-A2F treated group. Black Circles show H-A2F treated group. Open diamonds show A-84865-A2G2S2 treated group. Black triangles show A-8486-A2G2 treated group.

FIG. 11 shows that an antibody displaying A2 Glycan structure is distributed partially to the liver area with a fast kinetic as compared to control antibodies. Mice were injected i.v with CF750-labeled antibodies at 5 mg/kg and imaged using fluorescence tomography. The graph shows the average fluorescence in pmol±SD of 3 animals/time point in the gated liver region of interest. Open Squares and dotted line show Ptz-A2F treated group. Black Circles show H-A2F treated group. Open diamonds show A-84865-A2G2S2 treated group. Black triangles show A-8486-A2 treated group.

### DETAILED DESCRIPTION

Described herein is a glycoengineered bifunctional binding protein (e.g., a GlcNAc glycosylated bifunctional binding protein) having improved functionalities as compared to a control antibody. As exemplified herein, the glycoengineered bifunctional binding protein is engineered by introduction of glycosylation sites on the glycoengineered bifunctional binding protein, resulting in an engineered glycosylation profile that mediates endocytic receptor degradation of the glycoengineered bifunctional binding protein and the target to which it binds. By customizing the N-glycosylation, the glycoengineered bifunctional binding proteins described herein: 1) have homogeneous glycosylation; 2) can degrade large targets such as immune complexes; 3) have a defined ligand-to-antibody ratio; 4) have defined glycosylation sites; 6) can activate more diverse and powerful degradation receptors; and/or 6) can engage in protein degradation in a highly optimized manner.

Without being bound by theory, glycan engagement with endocytic carbohydrate-binding proteins and receptors enables different biological pathways. These essential biological pathways are involved in modulating immune responses, mediating protein clearance, protein turnover, and controlling trafficking of soluble glycoproteins, glycolipids and any natural molecule containing a glycan moiety. The glycan-receptor interaction is determined by the glycan structure. Glycan binding receptors are highly diverse and can be exploited by glycoengineering to develop novel therapeutics based on the concept of glycan-mediated protein degradation to treat different diseases, which include but are not limited to inflammatory disorders, blood disorders, autoimmune disorders, infectious diseases, and cancer.

Lectin receptors are involved in glycan-mediated endocytosis. Specifically, lectins capture glycoproteins via specific glycan structures to mediate lysosomal degradation. These endocytic lectins are ubiquitous in human and can be found on different cells.

As used herein the term “glycan” can refer to an N-glycan. Based on the specific structure, the skilled artisan would know if a specific glycan is an N-linked glycan.

Without being bound by theory, the glycoengineered bifunctional binding protein, as described herein, is expected to activate natural degradation pathways. Thus, the glycoengineered bifunctional binding protein is expected to reduce target proteins that are associated with human disease. Further, the glycoengineered bifunctional binding protein can be a GlcNAc glycosylated antibody, as described herein.

The term “about,” when used in conjunction with a number, refers to any number within +1, +5 or +10% of the referenced number.

As used herein, the term “patient” refers to an animal (e.g., birds, reptiles, and mammals). In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, a subject is a non-human animal. In some embodiments, a subject is a farm animal or pet (e.g., a dog, cat, horse, goat, sheep, pig, donkey, or chicken). In a specific embodiment, a subject is a human. The terms “subject” and “patient” can be used herein interchangeably.

The abbreviations “a [number]”, “a [number], [number]”, “B [number]”, or “B [number], [number]” refer to glycosidic bonds or glycosidic linkages which are covalent bonds that join a carbohydrate residue to another group. An α-glycosidic bond is formed when both carbons have the same stereochemistry, whereas a β-glycosidic bond occurs when the two carbons have different stereochemistry.

As used herein, the term “glycoengineering,” “glycoengineered,” or an equivalent thereof means a process of glycosylating a target protein, or a target protein (e.g., bifunctional binding protein) made by such process, wherein the process uses an in vivo host cell system that has one or more enzymes (e.g., pathways) that provides for glycosylation of the target protein. Such a host cell system can be genetically engineered to introduce a glycosylation pathway to selectively glycosylate a target protein with a particular glycan structure. A host cell used to generate a glycoengineered target protein can include, for example, a recombinant nucleic acid encoding a target protein; and a recombinant nucleic acid encoding a heterologous glycosyltransferase. The host cell system used for glycoengineering (e.g., to generate a glycoengineered protein) can introduce N-linked glycosylation. The host cell used for glycoengineering or to generate a glycoengineered target protein can be a mammalian cell, an insect cell, a yeast cell, a bacterial cell, a plant cell, a microalgae, or a protozoa. The protozoa used for glycoengineering can be a species of Leishmania. A glycoengineered target protein also includes a target protein that has been engineered to be selectively glycosylated at one or more specific sites when generated in the host cell system.

As used herein, the term “glycosite” or “glycosylation site” refers to a site of glycosylation in a protein. Such a glycosite can be naturally present in the amino acid sequence of a protein or recombinantly engineered into the protein by addition or substitution or deletion of amino acids. In a specific embodiment, a glycosite is present in a so-called glycotag that is fused to a bifunctional protein provided herein. In certain embodiments, a glycotag is fused to a protein to create a bispecific binding protein. As used herein a glycotag refers to a peptide containing consensus N-glycosylation site sequence fused to N- or a C-terminal or both termini of a protein or polypeptide. In some embodiments, the glycotag is fused to the C-terminus of the of the bifunctional protein via a peptide linker. In some embodiments, the glycotag is fused to the N-terminus of the bifunctional protein via a peptide linker. In some embodiments, the peptide linker is a consensus peptide sequence. In some embodiments, the consensus peptide sequence is 1, 2, 3, 4, 5, 6, 7 or more amino acid residues in length. In some embodiments, the bifunctional protein provided herein contains 1, 2, 3, 4, 5, 6, 7, 8, 9, 10 or more glycotags.

As used herein, the term “inflammatory disorder” includes disorders, diseases or conditions characterized by inflammation. Examples of inflammatory disorders include allergy, asthma, autoimmune diseases, coeliac disease, glomerulonephritis, hepatitis, inflammatory bowel disease, reperfusion injury and transplant rejection, among others.

As used herein, the term “blood disorder” includes a disorders, diseases or conditions that affect blood. Examples of blood disorders include anemia, bleeding disorders such as hemophilia, blood clots, and blood cancers such as leukemia, lymphoma, and myeloma, among others.

As used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin.

In one aspect, provided herein is a glycoengineered bifunctional binding protein comprising a first moiety that specifically binds to a target protein and a second moiety comprising a glycan comprising terminal GlcNAc.

In one aspect, provided herein is a glycoengineered bifunctional binding protein comprising a first moiety that specifically binds to a target protein and a second moiety comprising a glycan comprising terminal GalNAc.

In one aspect, provided herein is a glycoengineered bifunctional binding protein comprising a first moiety that specifically binds to a target protein and a second moiety comprising a glycan comprising terminal Gal.

In some embodiments, a glycoengineered bifunctional binding protein provided herein can comprise (i) a binding specificity to one or more target protein(s) and (ii) one or more N-glycan(s) with binding specificities to one or more endocytic carbohydrate-binding protein(s) or receptor(s). In some embodiments, the glycoengineered bifunctional binding protein has one binding specificity to one target protein. In more specific embodiments, the glycoengineered bifunctional binding protein has one binding specificity to one target protein and has 1, 2, 3, 4, 5, 6, 7, 8, 9, 10 or more valences so that a single bifunctional binding protein can bind to 1, 2, 3, 4, 5, 6, 7, 8, 9, 10 or more target protein molecules. In some embodiments, the glycoengineered bifunctional binding protein has two binding specificities to two different target proteins. In more specific embodiments, the glycoengineered bifunctional binding protein has: (i) one binding specificity to one target protein and has 1, 2, 3, 4, 5, 6, 7, 8, 9, 10 or more valences so that a single bifunctional binding protein can bind to 1, 2, 3, 4, 5, 6, 7, 8, 9, 10 or more target protein molecules; and (ii) another binding specificity to another target protein and has 1, 2, 3, 4, 5, 6, 7, 8, 9, 10 or more valences so that a single bifunctional binding protein can bind to 1, 2, 3, 4, 5, 6, 7, 8, 9, 10 or more other target protein molecules. In some embodiments, the glycoengineered bifunctional binding protein has three binding specificities to three target proteins. In more specific embodiments, the glycoengineered bifunctional binding protein has (i) a first binding specificity to a first target protein and has 1, 2, 3, 4, 5, 6, 7, 8, 9, 10 or more valences so that a single bifunctional binding protein can bind to 1, 2, 3, 4, 5, 6, 7, 8, 9, 10 or more first target protein molecules; (ii) a second binding specificity to a second target protein and has 1, 2, 3, 4, 5, 6, 7, 8, 9, 10 or more valences so that a single bifunctional binding protein can bind to 1, 2, 3, 4, 5, 6, 7, 8, 9, 10 or more second target protein molecules; and (iii) a third binding specificity to a third target protein and has 1, 2, 3, 4, 5, 6, 7, 8, 9, 10 or more valences so that a single bifunctional binding protein can bind to 1, 2, 3, 4, 5, 6, 7, 8, 9, 10 or more third target protein molecules.

In some embodiments, the glycoengineered bifunctional binding protein comprises one type of N-glycan with binding specificity to one type of endocytic carbohydrate-binding protein or receptor. In more specific embodiments, the glycoengineered bifunctional binding comprises 1, 2, 3, 4, 5, 6, 7, 8, 9, 10 or more N-glycosylation sites (or glycosites; such as an N-glycosylation consensus sequence). These N-glycosylation sites can be glycosylated by an N-glycan such that the resulting glycoengineered bifunctional binding protein can engage with or bind to 1, 2, 3, 4, 5, 6, 7, 8, 9, 10 or more endocytic carbohydrate-binding protein molecules or receptor molecules.

In some embodiments, the glycoengineered bifunctional binding protein comprises types of two N-glycans with binding specificities to two different endocytic carbohydrate-binding proteins or receptors. In certain embodiments, a glycoengineered bifunctional binding protein provided herein can comprise 1, 2, 3, 4, 5, 6, 7, 8, 9, 10 or more polypeptide chains. Each chain can be produced in a different cell line. In certain embodiments, the glycoengineered bifunctional binding protein can be an antibody and one type of N-glycan is on the Fc domain and another type of N-glycan is on the Fab domain (eg, the variable regions) of the antibody.

In more specific embodiments, the glycoengineered bifunctional binding protein comprises: (i) a first type of N-glycan with binding specificity to a first endocytic carbohydrate-binding protein or receptor wherein the first type of N-glycan is present at 1, 2, 3, 4, 5, 6, 7, 8, 9, 10 or more glycosites thus engaging with or binding to 1, 2, 3, 4, 5, 6, 7, 8, 9, 10 or more molecular of the first endocytic carbohydrate-binding protein or receptor; and (ii) a second type of N-glycan with binding specificity to a second endocytic carbohydrate-binding protein or receptor wherein the second N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the second endocytic carbohydrate-binding protein(s) or receptor(s).

In more specific embodiments, the glycoengineered bifunctional binding protein comprises: (i) a first type of N-glycan with binding specificity to a first endocytic carbohydrate-binding protein or receptor wherein the first type of N-glycan is present at 1, 2, 3, 4, 5, 6, 7, 8, 9, 10 or more glycosites thus engaging with or binding to 1, 2, 3, 4, 5, 6, 7, 8, 9, 10 or more molecular of the first endocytic carbohydrate-binding protein or receptor; (ii) a second type of N-glycan with binding specificity to a second endocytic carbohydrate-binding protein or receptor wherein the second N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the second endocytic carbohydrate-binding protein(s) or receptor(s); and (iii) a third type of N-glycan with binding specificity to a third endocytic carbohydrate-binding protein or receptor wherein the third N-glycan is present at 1, 2, 3, 4, 5, 6, 7, 8, 9, 10 or more glycosites so that a single bifunctional binding protein can engage with or bind to 1, 2, 3, 4, 5, 6, 7, 8, 9, 10 or more molecules of the third endocytic carbohydrate-binding protein(s) or receptor(s).

In some embodiments, a glycoengineered bifunctional binding protein provided herein has 1, 2, 3, 4, 5, 6, 7, 8, 9, 10 or more glycosites. In some embodiments, in a population of glycoengineered bifunctional binding proteins, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycosites in the population at one specific position are glycosylated. In certain embodiments, in a population of glycoengineered bifunctional binding proteins, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycosites in the population are glycosylated. N-glycans that can be present at the glycosites of the glycoengineered bifunctional binding protein provided herein are described below.

In certain embodiments, a glycosite is an N-glycosylation consensus sequence. The consensus sequence can be N-X-S/T, or N-X-C, wherein X is any amino acid except proline.

In some embodiments, provided herein is a glycoengineered bifunctional binding protein, that specifically binds to a target protein associated with a disease, comprising a first moiety and a second moiety. In some embodiments, provided herein is a glycoengineered bifunctional binding protein comprising a first moiety that specifically binds to a target protein associated with a disease and a second moiety that binds specifically to an endocytic carbohydrate-binding protein or receptor, wherein the second moiety comprises a glycan structure.

In some embodiments, provided herein is a glycoengineered bifunctional binding protein comprising a first moiety that specifically binds to a target protein and a second moiety comprising an N-glycan selected from the group consisting of GlcNAc2Man3GlcNAc2, GalNAc2GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

In some embodiments, provided herein is a glycoengineered bifunctional binding protein comprising a first moiety that specifically binds to a target protein and a second moiety comprising a glycan comprising the structure:

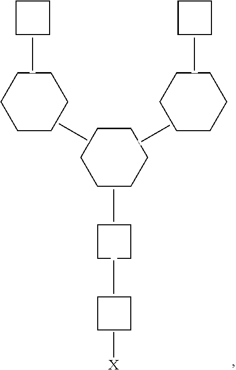

wherein the square represents an N-acetylglucosamine (GlcNAc) residue and the hexagon represents a mannose (Man) residue, and wherein X represents an amino acid residue of the bifunctional binding protein. For naming of this structure see Table 1.

In some embodiments, provided herein is a glycoengineered bifunctional binding protein comprising a first moiety that specifically binds to a target protein and a second moiety comprising a glycan comprising the structure:

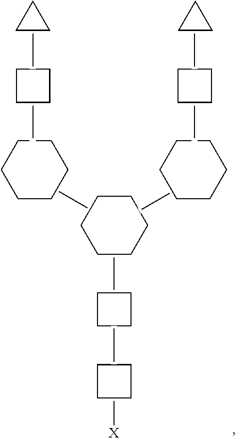

wherein the triangle represents an N-acetyl galactosamine (GalNAc), the white square represents an N-acetylglucosamine (GlcNAc) residue and the hexagon represents a mannose (Man) residue, and wherein X represents an amino acid residue of the bifunctional binding protein. For naming of this structure see Table 1.

In some embodiments, provided herein is a glycoengineered bifunctional binding protein comprising a first moiety that specifically binds to a target protein and a second moiety comprising a glycan comprising the structure:

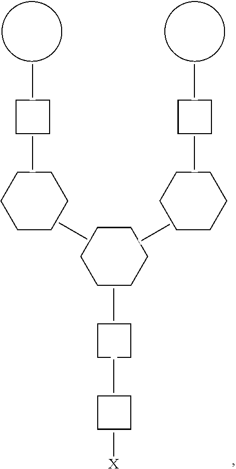

wherein the white circle represents a galactose (Gal) residue, the square represents an N-acetylglucosamine (GlcNAc) residue and the hexagon represents a mannose (Man) residue, and wherein X represents an amino acid residue of the bifunctional binding protein. For naming of this structure see Table 1.

In some embodiments, provided herein is a glycoengineered bifunctional binding protein, wherein the bifunctional binding protein (i) specifically binds to a target protein and (ii) comprises an N-glycan of the structure:

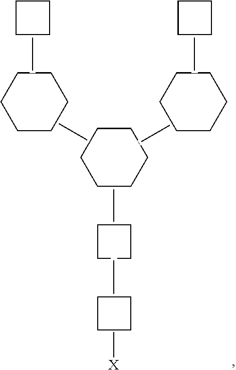

wherein the N-glycan is linked to the bifunctional binding protein at 1, 2, 3, 4 or 5 N-glycosylation sites. For naming of this structure see Table 1. In certain embodiments, a bifunctional protein provided herein comprises a N-glycan with a GlcNAc2 as the terminal glycan. Specifically, any branched structure of the N-glycan on the GlcNAc2 part of the N-glycan can also be included.

In some embodiments, provided herein is a glycoengineered bifunctional binding protein, wherein the bifunctional binding protein (i) specifically binds to a target protein and (ii) comprises an N-glycan of the structure:

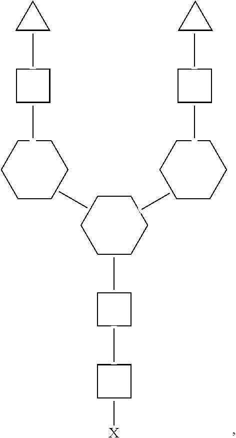

wherein the N-glycan is linked to the bifunctional binding protein at 1, 2, 3, 4 or 5 N-glycosylation sites. For naming of this structure see Table 1. In certain embodiments, a bifunctional protein provided herein comprises a N-glycan with a GalNAc2 as the terminal glycan. Specifically, any branched structure of the N-glycan on the GalNAc2 part of the N-glycan can also be included.

In some embodiments, provided herein is a glycoengineered bifunctional binding protein, wherein the bifunctional binding protein (i) specifically binds to a target protein and (ii) comprises an N-glycan of the structure:

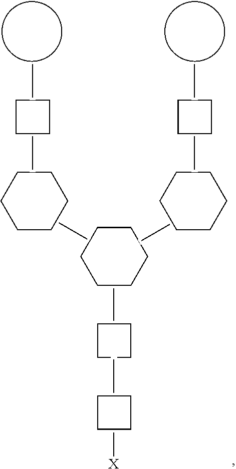

wherein the N-glycan is linked to the bifunctional binding protein at 1, 2, 3, 4 or 5 N-glycosylation sites. For naming of this structure see Table 1. In certain embodiments, a bifunctional protein provided herein comprises a N-glycan with a Gal2 as the terminal glycan. Specifically, any branched structure of the N-glycan on the Gal2 part of the N-glycan can also be included

In some embodiments, the glycoengineered bifunctional binding protein is an antibody or a fragment thereof. In some embodiments, the Fab region of the antibody has 1, 2, 3, 4, 5, 6, 7, 8, 9, 10 or more glycosites. In other embodiments, the 1, 2, 3, 4, 5, 6, 7, 8, 9, 10 or more glycosites in the Fab region are linked to an N-glycan structure, for example, any of the glycan structures shown in Table 11. In certain embodiments, the glycan has one of the following structures:

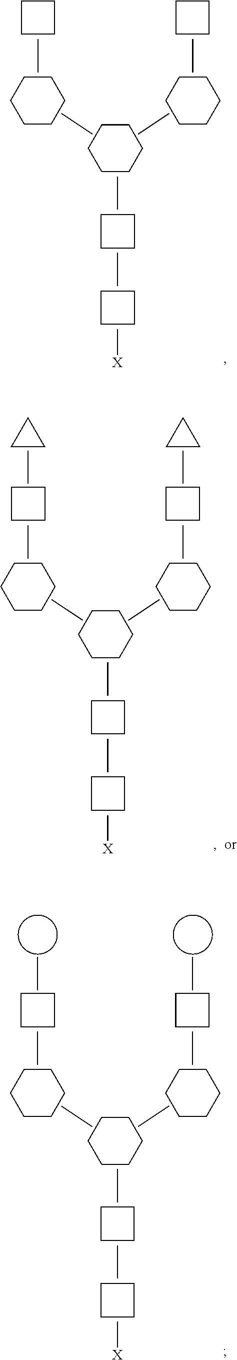

wherein the N-glycan is linked to the bifunctional binding protein at 1, 2, 3, 4 or 5 N-glycosylation sites, and wherein the triangle represents an N-acetyl galactosamine (GalNAc), the white square represents an N-acetylglucosamine (GlcNAc) residue and the hexagon represents a mannose (Man) residue, the white circle represents a galactose (Gal) residue, and wherein X represents an amino acid residue of the bifunctional binding protein. For naming of these structures, see Table 1.

In some embodiments, the Fc region of the antibody has 1, 2, 3, 4, 5, 6, 7, 8, 9, 10 or more glycosites. In certain embodiments, 1, 2, 3, 4, 5, 6, 7, 8, 9, 10 or more of these glycosites comprise an N-glycan structure, for example, any of the glycan structures shown in Table 11. In certain embodiments, the glycan has one of the following structures:

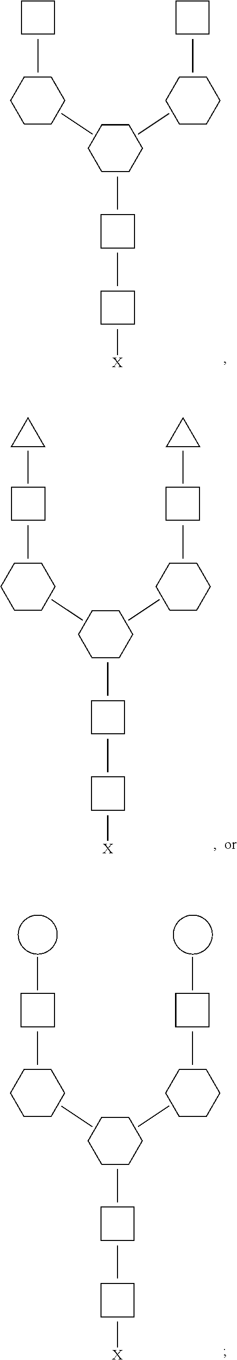

wherein the N-glycan is linked to the bifunctional binding protein at 1, 2, 3, 4 or 5 N-glycosylation sites, and wherein the triangle represents an N-acetyl galactosamine (GalNAc), the white square represents an N-acetylglucosamine (GlcNAc) residue and the hexagon represents a mannose (Man) residue, the white circle represents a galactose (Gal) residue, and wherein X represents an amino acid residue of the bifunctional binding protein. For naming of these structures, see Table 1.

In some embodiments, the Fab region and the Fc region each has 1, 2, 3, 4, 5, 6, 7, 8, 9, 10 or more glycosites, wherein 1, 2, 3, 4, 5, 6, 7, 8, 9, 10 or more of these glycosites are glycosylated with an N-glycan for example, any of the glycan structures shown in Table 11. In certain embodiments, the glycan has one of the following structures:

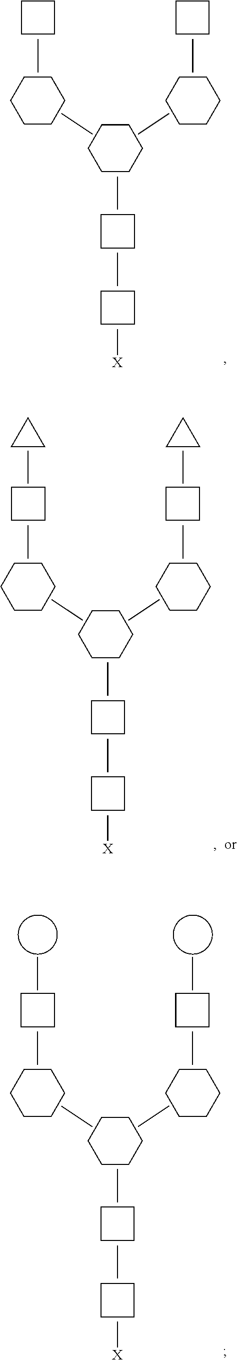

wherein the N-glycan is linked to the bifunctional binding protein at 1, 2, 3, 4 or 5 N-glycosylation sites, and wherein the triangle represents an N-acetyl galactosamine (GalNAc), the white square represents an N-acetylglucosamine (GlcNAc) residue and the hexagon represents a mannose (Man) residue, the white circle represents a galactose (Gal) residue, and wherein X represents an amino acid residue of the bifunctional binding protein. For naming of these structures, see Table 1.

In certain embodiments, any branched structure of the N-glycan of the proximal GlcNAc (the GlcNAc fused to the glycoengineered bifunctional binding protein) can also be included. In certain embodiments, the proximal GlcNAc can be fucosylated. In certain embodiments, the N-glycan consists of any one of the structures shown above.

In some embodiments, the Fab region contains more glycans than the Fc region. In some embodiments, the Fab region contains 1, 2, 3, 4, 5, 6, 7, 8, 9, 10 or more glycans compared to the Fc region. In some embodiments, 10% of the glycans are in the Fc region and 90% of the glycans are in the Fab region. In some embodiments, 20% of the glycans are in the Fc region and 80% of the glycans are in the Fab region. In some embodiments, 30% of the glycans are in the Fc region and 70% of the glycans are in the Fab region. In some embodiments, 40% of the glycans are in the Fc region and 60% of the glycans are in the Fab region. In some embodiments, 50% of the glycans are in the Fc region and 50% of the glycans are in the Fab region. In some embodiments, the glycan structures in the Fab region and Fc region are identical (i.e., the same). In some embodiments, the glycan structures in the Fab region and Fc region are nonidentical (i.e., not the same).

In some embodiments, the first moiety comprises a heavy chain variable region and a light chain variable region, or a functional fragment thereof. In some embodiments, the first moiety comprises a Fab region of a monoclonal antibody. In some embodiments, the first moiety specifically binds to any of the target proteins disclosed herein.

In some embodiments, the glycoengineered bifunctional binding protein is a TNFα monoclonal antibody. In other embodiments, the glycoengineered bifunctional binding protein is not a TNFα monoclonal antibody.

In some embodiments, the second moiety comprises a glycan structure. In some embodiments, the glycan structure comprises any one of the glycan structures disclosed in Table 1. In some embodiments, the glycan structure comprises GlcNAc2Man3GlcNAc2, GalNAc2GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan. In some embodiments, the glycan structure is a GlcNAc2Man3GlcNAc2 structure as disclosed herein. In some embodiments, the glycan structure is a GalNAc2GlcNAc2Man3GlcNAc2 structure as disclosed herein. In some embodiments, the glycan structure is a Gal2GlcNAc2Man3GlcNAc2 structure as disclosed herein. In some embodiments, the glycan structure is a mannose 3 glycan structure as disclosed herein. In some embodiments, the glycan structure is any of the glycans disclosed herein.

In some embodiments, the second moiety specifically binds to an endocytic lectin. In some embodiments, the endocytic lectin is a mannose binding receptor. In some embodiments, the endocytic lectin is a Cluster of Differentiation 206 (CD206) receptor. In some embodiments, the endocytic lectin is a DC-SIGN (Cluster of Differentiation 209 or CD209) receptor. In some embodiments, the endocytic lectin is a C-Type Lectin Domain Family 4 Member G (LSECTin) receptor. In some embodiments, the endocytic lectin is a macrophage inducible Ca²⁺-dependent lectin receptor (Mincle). In some embodiments, the endocytic receptor is L-SIGN CD 209L). In some embodiments, the endocytic receptor is asialoglycoprotein (ASGPR). In some embodiments, the endocytic receptor is dectin-1. In some embodiments, the endocytic receptor is dectin-2. In some embodiments, the endocytic receptor is langerin. In some embodiments, the second moiety specifically binds to a receptor selected from the group consisting of macrophage mannose 2 receptor, BDCA-2, DCIR, MBL, MDL, MICL, CLEC2, DNGR1, CLEC12B, DEC-205, and mannose 6 phosphate receptor (M6PR).

CD206 is a C-type lectin and phagocytic/endocytic recycling and signaling receptor. CD206 is expressed primarily by M2 anti-inflammatory macrophages, dendritic cells, and live sinusoidal endothelial cells. DC-SIGN is a non-recycling, signaling receptor that targets both the ligand and receptor to the lysosome for degradation. LSECTin is expressed on liver sinusoidal endothelial cells.

ASGPR-mediated degradation in the hepatocyte has many applications. ASPGR binding to glycan structures disclosed herein can result in the selective degradation of soluble or cell surface proteins. ASGPR-mediated degradation can lead to removal of cytokines, chemokines and hormones. Additionally, ASGPR-mediated degradation can be used for the delivery of active molecules to the hepatocyte endosome. Thus, making ASGPR-mediated degradation applicable for various liver diseases, while limiting systemic toxicity.

Without being bound by theory, in some embodiments, the glycan structures provided herein are bound by the receptors provided in Table 1. In some embodiments, any receptor that binds a GlcNAc2Man3GlcNAc2 glycan is included in the description of the compositions and methods disclosed herein. In some embodiments, any receptor that binds a GalNAc2GlcNAc2Man3GlcNAc2 glycan is included in the description of the compositions and methods disclosed herein. In some embodiments, any receptor that binds a Gal2GlcNAc2Man3GlcNAc2 structure is included in the description of the compositions and methods disclosed herein. In some embodiments, any receptor that binds a mannose 3 glycan is included in the description of the compositions and methods disclosed herein. In other embodiments, the second moiety binds to any endocytic carbohydrate-binding receptor recognizing Man3GlcNAc2 structure.

TABLE 1
Glycan moieties of the present disclosure and possible examples of respective
endocytic lectin receptors

| | | Symbol | |
|---|---|---|---|
| | Short IgG | Nomenclature for | |
| Oxford/Unifi | nomenclature² or | Glycan | Glycan |
| nomenclature¹ | alternative name | structure³ | receptor |
| M3 | Man3 | 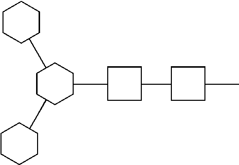 | CD206, MBL, Langerin, LSECTin, Mincle |
| A1 | G0-N | 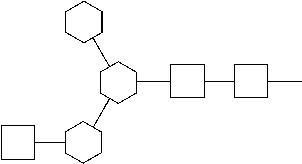 | GlcNAcR |
| A1G1 | G1-N | 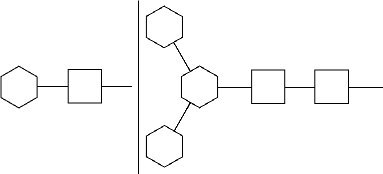 | Galectins ASGPR |
| A1[3]G(4)1 | G1-N | 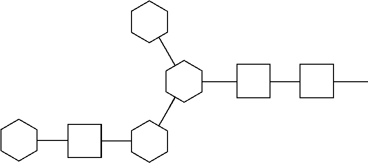 | Galectins, ASGPR |
| A2 | G0 | 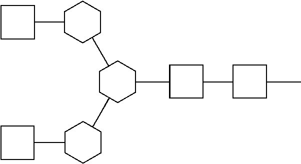 | LSECtin, GlcNAcR CD206, DC-SIGN |
| A2[6]G(4)1 | G1(6′) | 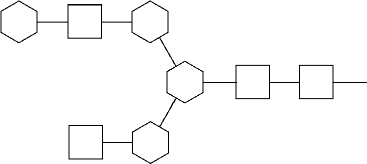 | Galectins, ASGPR, DC-SIGN |
| A2[3]G(4)1 | G1(3′) | 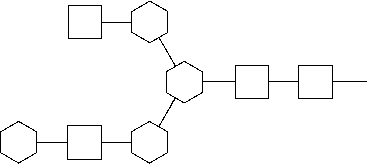 | Galectins, ASGPR |
| A2G1 | G1 | 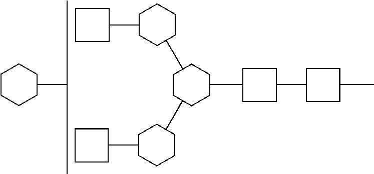 | Galectins ASGPR |
| | | 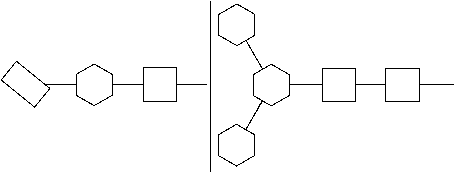 | |
| | | 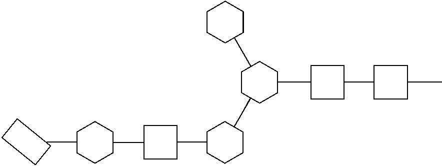 | |
| A2G(4)2 | G2 | 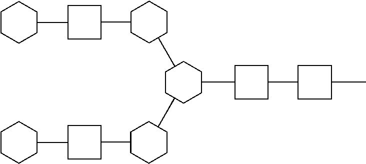 | Galectins, ASGPR, BDCA-2 |
| A2G1S1 | G1S1 | 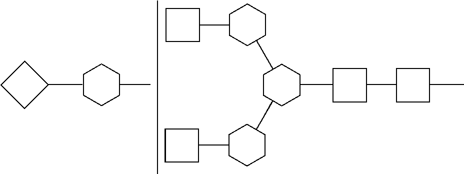 | , LSECTin |
| A2[6]G(4)1S(6)1 | G1S1 | 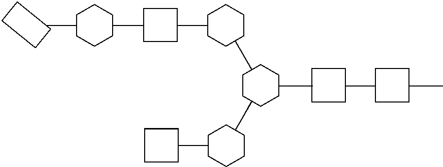 | LSECTin |
| A2[3]G(4)1S(6)1 | G1S1 | 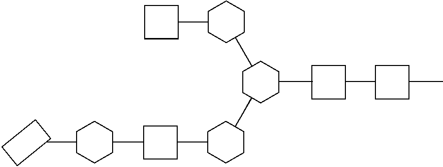 | , LSECTin |
| | | 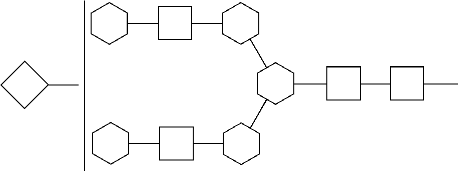 | |
| | | 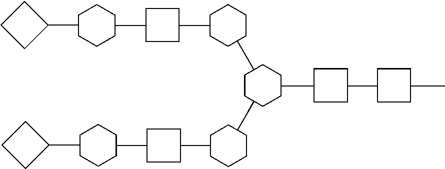 | |
| | | 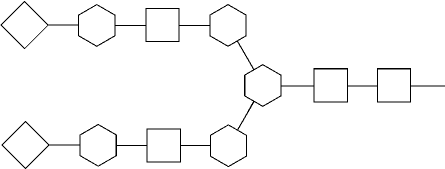 | |
| M4 | Man4 | 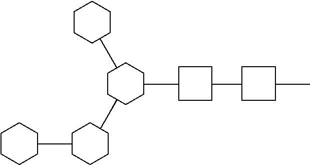 | CD206 |
| M5 | Man5 (note = other isoform on IgG) | 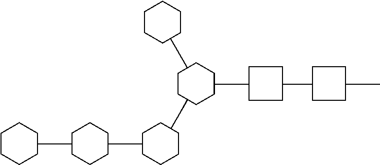 | CD206, Langerin |
| M6 | Man6 | 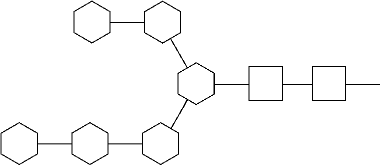 | CD206, Langerin |
| M7 | Man7 | 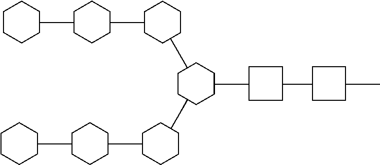 | CD206, Langerin |
| M8 | Man8 | 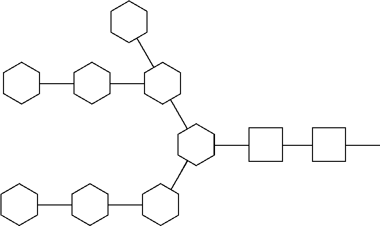 | CD206, Langerin |
| M9 | Man9 | 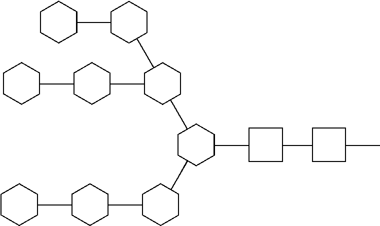 | CD206, DC-SIGN, Langerin |
| A3(2,2,3) | A3 or Tri-antennary GlcNAc or Tri- GlcNAc | 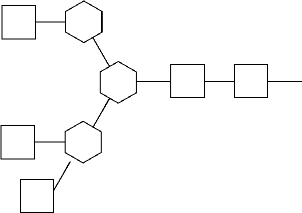 | GlcNAcR |
| A3(2,2,6) | A3 or Tri-antennary GlcNAc or Tri-GlcNAc | 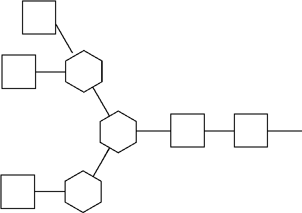 | GlcNAcR |
| A4 | A4 or Tetra-antennary GlcNAc or Tetra-GlcNAc | 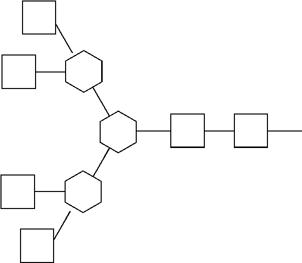 | GlcNAcR |
| A1[3]GalNAc(4)1 | A1GalNAc1 or Mono-antennary GalNAc | 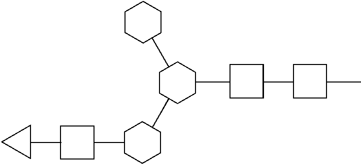 | ASGPR |
| A2[3](2)GalNAc(4)1 or A2[6](2)GalNAc(4)1 | A2GalNAc1 | 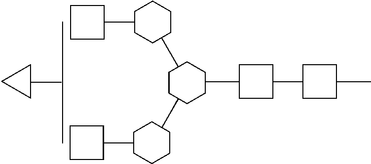 | ASGPR |
| A2GalNAc(4)2 | A2GalNAc2 or Bi-antennary GalNAc or Bi-GalNAc | 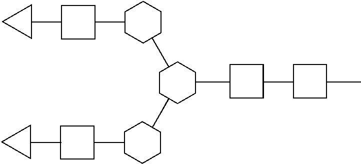 | ASGPR |
| A3(2,2,4)GalNAc(4)3 | A3GalNAc3 or Tri-antennary GalNAc or Tri-GalNAc | 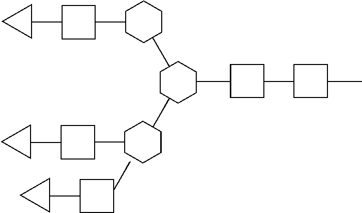 | ASGPR |
| A3(2,2,6)GalNAc(4)3 | A3GalNAc3 or Tri-antennary GalNAc or Tri-GalNAc | 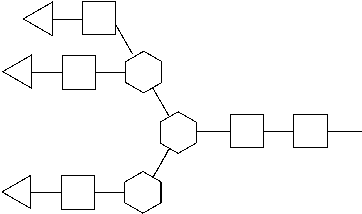 | ASGPR |
| A4GalNAc(4)4 | A4GalNAc4 or Tetra-antennary GalNAc or Tetra-GalNAc | 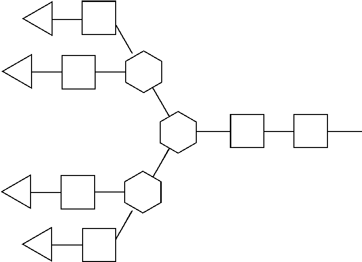 | ASGPR |
| A3(2,2,4)G(4)3 | G3 or Tri-antennary Gal or Tri-Gal | 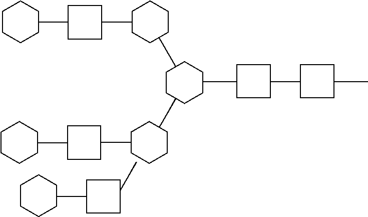 | ASGPR |
| A3(2,2,6)G(4)3 | G3 or Tri-antennary Gal or Tri-Gal | 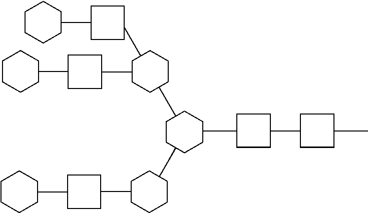 | ASGPR |
| A4(4)4 | G4 or Tetra-antennary Gal or Tetra-Gal | 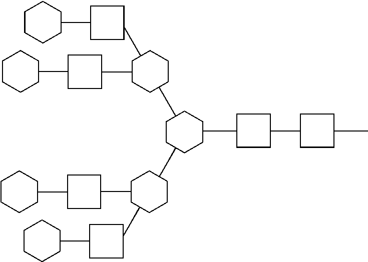 | ASGPR |
| | | 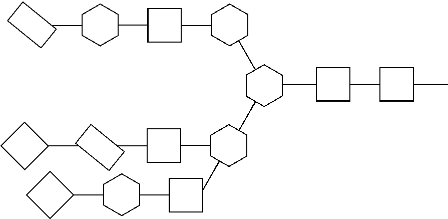 | |
| | | 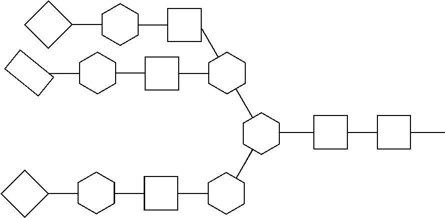 | |
| | | 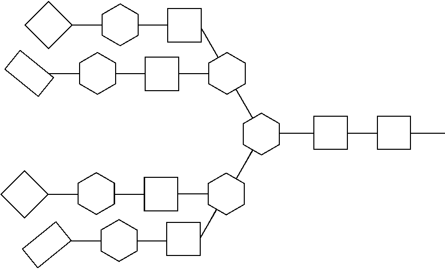 | |
| M6P glycans: phosphomonoesters | Man3(-P)n | 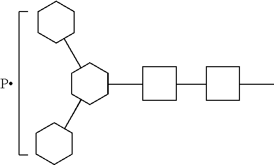 | M6PR |
| | Man4(-P)n | 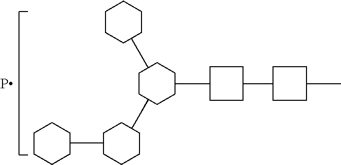 | M6PR |
| | Man5(-P)n | 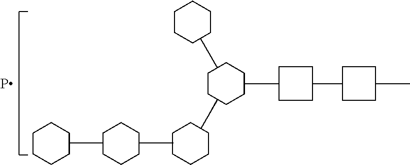 | M6PR |
| | Man6(-P)n | 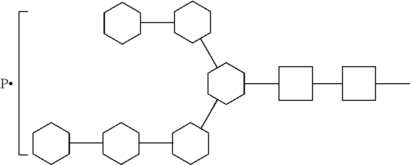 | M6PR |
| | Man7(-P)n | 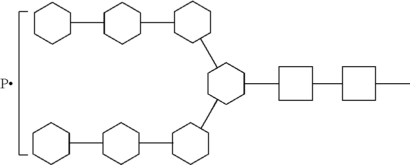 | M6PR |
| | Man8(-P)n | 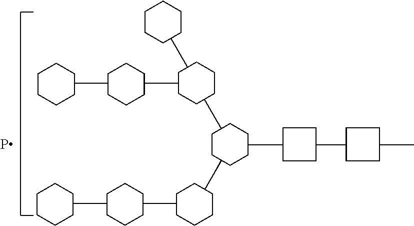 | M6PR |
| | Man9(-P)n | 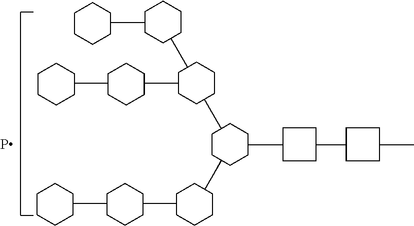 | M6PR |
| M6P glycans: phosphodiesters | Man3(-P-Man)n | 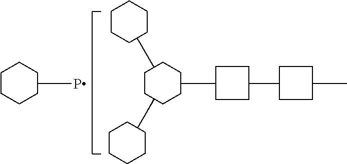 | M6PR |
| | Man4(-P-Man)n | 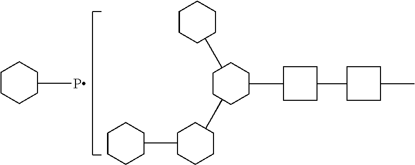 | M6PR |
| | Man5(-P-Man)n | 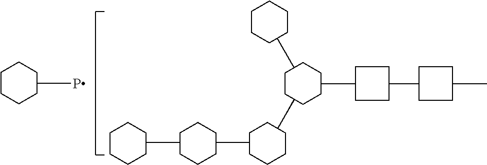 | M6PR |
| | Man6(-P-Man)n | 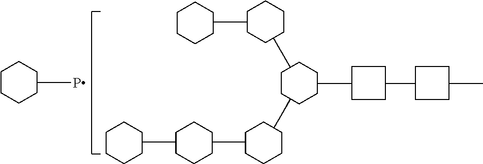 | M6PR |
| | Man7(-P-Man)n | 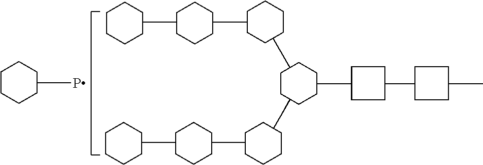 | M6PR |
| | Man8(-P-Man)n | 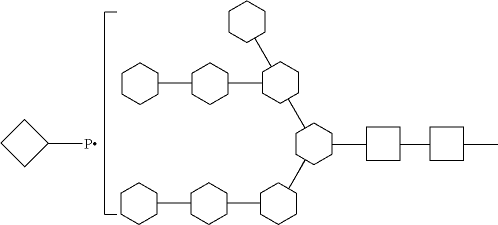 | M6PR |
| | Man9(-P-Man)n | 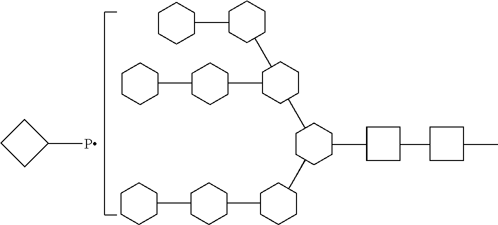 | M6PR |
| | Man3(-P-GlcNAc)n | 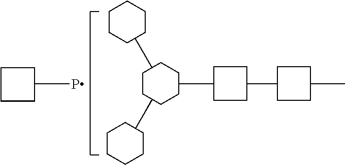 | M6PR |
| | Man4(-P--GlcNAc)n | 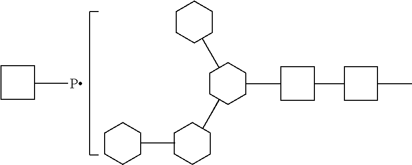 | M6PR |
| | Man5(-P-GlcNAc)n | 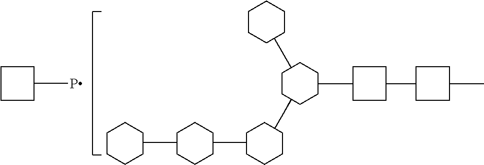 | M6PR |
| | Man6(-P-GlcNAc)n | 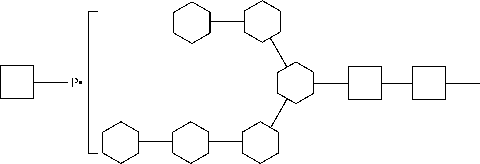 | M6PR |
| | Man7(-P-GlcNAc)n | 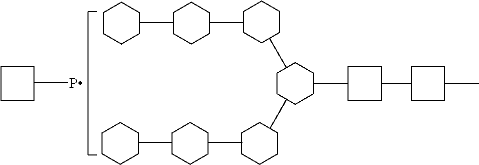 | M6PR |
| | Man8(-P-GlcNAc)n | 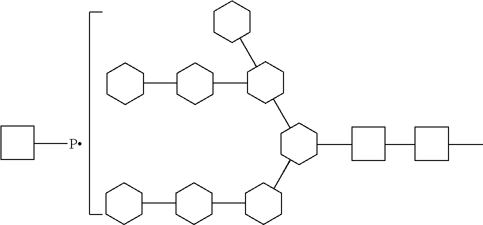 | M6PR |
| | Man9(-P-GlcNAc)n | 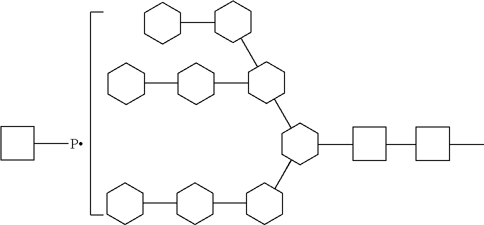 | M6PR |

¹Mx: number (x) of residues within the oligomannose series; Ax: number (x) of antennae; F: core fucose; Gx: number (x) of galactoses; B: bisecting GlcNAc; S: number (x) of sialic acids.
Note:
Linkage information is given in () parentheses if applicable, e.g. A2G1S1(6)-α2-6 linked sialic acid. Brackets [x] before G or GalNAc indicate which arm of the mannosyl core is galactosylated e.g. [3]G1 indicates that the galactose is on the antenna of the α1-3 mannose.
²This typically with IgG associated naming system indicates the presence of core fucose, the number of galactoses and the presence of bi-antennary glycans. It is limited in the number of structures and linkages it can describe but is often used for simplicity.
³hexagon represents mannose (Man), white square is N-acetyl glucosamine (GlcNAc), triangle is N-acetyl galactosamine (GalNAc), white circle is galactose (Gal), white diamond is sialic acid, N-acetyl neuraminic acid (Neu5Ac), P is phosphate.

In some embodiments, the glycoengineered bifunctional binding protein is an antibody. In some embodiments, the antibody is a monoclonal antibody, a polyclonal antibody, or functional fragment thereof. In some embodiments, the antibody is a recombinant antibody. In some embodiments, the antibody is isolated from a human subject. In some embodiments, the antibody is humanized, chimeric or fully human. In other embodiments, the glycoengineered bifunctional binding protein is an autoantigen. In other embodiments, the glycoengineered bifunctional binding protein is an autoantibody.

In some embodiments, the antibody has a glycan to antibody ratio of 2 to 1, 4 to 1, 6 to 1, 8 to 1 or 10 to 1. In some embodiments, the antibody is glycosylated at a predetermined and specific residue. In other embodiments, the antibody is glycosylated at a random residue.

In some embodiments, provided herein is a glycoengineered bifunctional binding protein comprising a second moiety with the following structure:

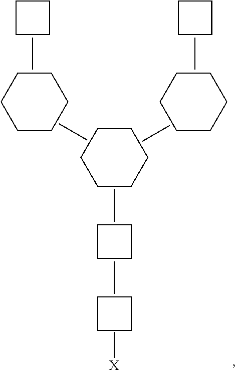

wherein the square represents an N-acetylglucosamine (GlcNAc) residue and the hexagon represents a mannose residue, and wherein X represents an amino acid residue of the glycoengineered bifunctional binding protein. In some embodiments, the X amino acid residue of the glycoengineered bifunctional binding protein as disclosed herein is an Asn of an N-linked glycosylation consensus sequence that is engineered into the glycoengineered bifunctional binding protein. Such an N-linked glycosylation consensus sequence can be, in some embodiments, in a variable domain of the glycoengineered bifunctional binding protein. In some embodiments, a population of the glycoengineered bifunctional binding protein comprises at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of glycans having the structure of

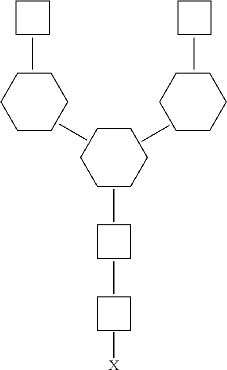

wherein X represents an amino acid residue of the glycoengineered bifunctional binding protein at a respective glycosite (symbols have the meaning introduced above). In some embodiments, the X amino acid residue of the glycoengineered bifunctional binding protein disclosed herein is an Asn of an N-linked glycosylation consensus sequence that is engineered into the glycoengineered bifunctional binding protein.

In some embodiments, provided herein is a glycoengineered bifunctional binding protein comprising a second moiety with the following structure:

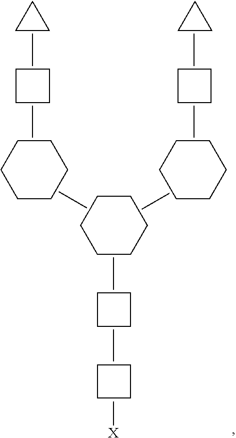

wherein the triangle represents N-acetylgalactosamine (GalNAc), the white square represents an N-acetylglucosamine (GlcNAc) residue and the hexagon represents a mannose residue, and wherein X represents an amino acid residue of the glycoengineered bifunctional binding protein. In some embodiments, the X amino acid residue of the glycoengineered bifunctional binding protein as disclosed herein is an Asn of an N-linked glycosylation consensus sequence that is engineered into the glycoengineered bifunctional binding protein. Such an N-linked glycosylation consensus sequence can be, in some embodiments, in a variable domain of the glycoengineered bifunctional binding protein. In some embodiments, a population of the glycoengineered bifunctional binding protein comprises at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycans having the
structure of

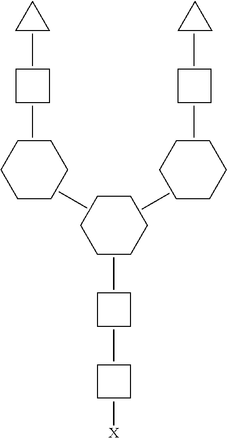

wherein X represents an amino acid residue of the glycoengineered bifunctional binding protein at a specific glycosite (symbols have the meaning introduced above). In some embodiments, the X amino acid residue of the glycoengineered bifunctional binding protein disclosed herein is an Asn of an N-linked glycosylation consensus sequence that is engineered into the glycoengineered bifunctional binding protein.

In some embodiments, provided herein is a glycoengineered bifunctional binding protein comprising a second moiety with the following structure:

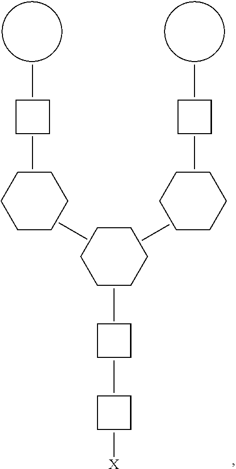

wherein the white circle represents galactose, the square represents an N-acetylglucosamine (GlcNAc) residue and the hexagon represents a mannose residue, and wherein X represents an amino acid residue of the glycoengineered bifunctional binding protein. In some embodiments, the X amino acid residue of the glycoengineered bifunctional binding protein as disclosed herein is an Asn of an N-linked glycosylation consensus sequence that is engineered into the glycoengineered bifunctional binding protein. Such an N-linked glycosylation consensus sequence can be, in some embodiments, in a variable domain of the glycoengineered bifunctional binding protein. In some embodiments, a population of the glycoengineered bifunctional binding protein comprises at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycans having the structure of

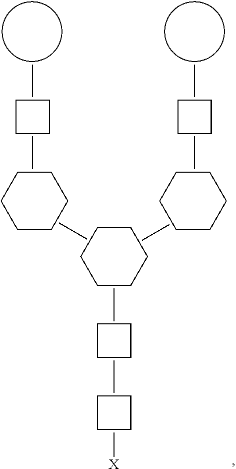

wherein X represents an amino acid residue of the glycoengineered bifunctional binding protein at a specific glycosite (symbols are as introduced above). In some embodiments, the X amino acid residue of the glycoengineered bifunctional binding protein disclosed herein is an Asn of an N-linked glycosylation consensus sequence that is engineered into the glycoengineered bifunctional binding protein.

In some embodiments, the glycoengineered bifunctional binding protein is an anti-TGF-β monoclonal antibody or an anti-Notch monoclonal antibody glycoengineered with Mannotriose-di-(N-acetyl-D-glucosamine) (Man3GlcNAc2). In some embodiments, the glycoengineered bifunctional binding protein is an anti-TNFα monoclonal antibody glycoengineered with GlcNAc2Man3GlcNAc2GalNAc2.

In other embodiments, provided herein is a glycoengineered bifunctional binding protein capable of binding to a specific target protein and comprising one or more of the following structures:

| | Short IgG | |
|---|---|---|
| | nomenclature² | Symbol Nomenclature for |
| | or alternative | Glycan |
| Oxford/Unifi nomenclature¹ | name | structure³ |
| M3 | Man3 | 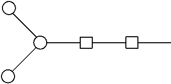 |
| A1 | G0-N | 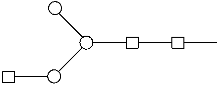 |
| A1G1 | G1-N | 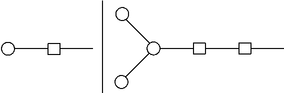 |
| A1[3]G(4)1 | G1-N | 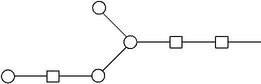 |
| A2 | G0 | 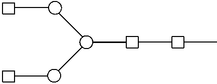 |
| A2[6]G(4)1 | G1(6′) | 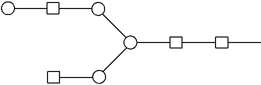 |
| A2[3]G(4)1 | G1(3′) | 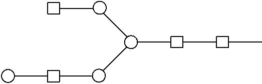 |
| A2G1 | G1 | 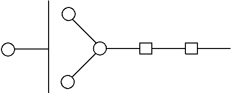 |
| A1G1S1 | G1S1-N | 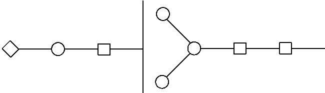 |
| A1[3]G(4)1S1 | G1S1-N | 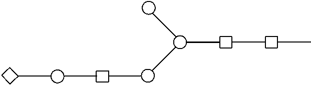 |
| A2G(4)2 | G2 | 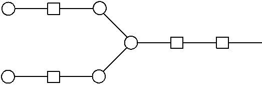 |
| A2G1S1 | G1S1 | 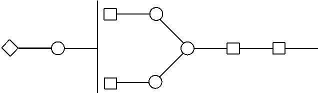 |
| A2[6]G(4)1S(6)1 | G1S1 | 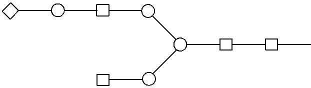 |
| A2[3]G(4)1S(6)1 | G1S1 | 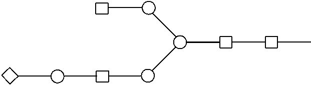 |
| A2G2S1 | G2S1 | 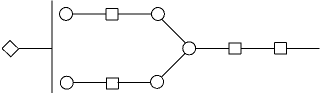 |
| A2G2S2 | G2S2 | 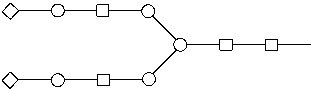 |
| A2G(4)2S(6,6)2 | G2S2 | 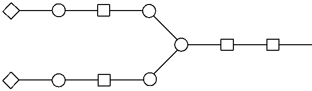 |
| A1[3]GalNAc(4)1 | A1GalNAc1 or Mono- antennary GalNAc | 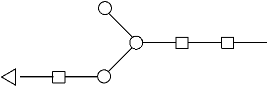 |
| A2[3](2)GalNAc(4)1 or A2[6](2)GalNAc(4)1 | A2GalNAc1 | 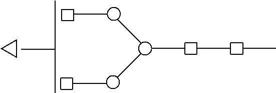 |
| A2GalNAc(4)2 | A2GalNAc2 or Bi-antennary GalNAc or Bi-GalNAc | 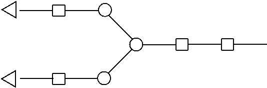 |
| A2F | G0F | 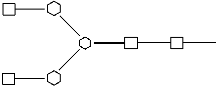 |
| A2G1F | G1F | 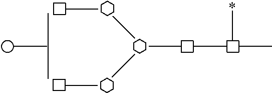 |
| A2G2S2F | G2FS2 | 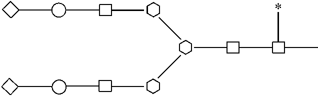 |

wherein, ¹Mx: number (x) of residues within the oligomannose series; Ax: number (x) of antennae; F: core fucose; Gx: number (x) of galactoses; B: bisecting GlcNAc; S: number (x) of sialic acids. Note: Linkage information is given in ( ) parentheses if applicable, e.g. A2G1S1(6)-α2-6 linked sialic acid. Brackets [x] before G or GalNAc indicate which arm of the mannosyl core is galactosylated e.g., [3]G1 indicates that the galactose is on the antenna of the α1-3 mannose. ²This typically with IgG associated naming system indicates the presence of core fucose, the number of galactoses and the presence of biantennary glycans. It is limited in the number of structures and linkages it can describe but is often used for simplicity. ³hexagon represents mannose (Man), white square is N-acetyl glucosamine (GlcNAc), triangle is N-acetyl galactosamine (GalNAc), white circle is galactose (Gal), white diamond is sialic acid, N-acetyl neuraminic acid (Neu5Ac) and asterisk is fucose (Fuc).

In some embodiments, the N-glycan present in a bispecific protein provided herein comprises Man3GlcNAc2, GlcNAc2Man3GlcNAc2, GlcNAc1Man3GlcNAc2, Gal2GlcNAc2Man3GlcNAc2, Gal1GlcNAc2Man3GlcNAc2, GalNAc2GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

In some embodiments, the target protein is a cell surface molecule or a non-cell surface molecule. In some embodiments, the cell surface molecule is a receptor. In some embodiments, the non-cell surface receptor is an extracellular protein. In some embodiments, the extracellular protein is an autoantibody, a hormone, a cytokine, a chemokine, a blood protein, or a protein expressed in the central nervous system (CNS).

In some embodiments, the target protein associated with a disease is upregulated in the disease compared to a non-disease state. In some embodiments, the target protein associated with a disease is expressed in the disease compared to a non-disease state. In some embodiments, the target protein associated with a disease is involved in the progression of the disease. In some embodiments, the disease is a cancer or tumor. In some embodiments, the target protein is involved in cancer progression. In some embodiments the disease is an autoimmune disease. In some embodiments, the disease is neurodegenerative disease.

In some embodiments, the disease is Graves' disease. Graves' disease is the most common cause of hyperthyroidism. Prevalence in the US is 1.2% (1), with lifetime risk in women as high as 3%. Production of agonistic anti-TSH Receptor (TSHR) antibodies (TRAb) leading to over production of thyroxine hormone (>90% of patients are TRAb+) (2). Current treatments have not advanced in the 50 years and are limited by high risk of recurrence or severe side effects such as hypothyroidism. In some embodiments, the target protein associated with Graves' disease is an autoantibody binding TSHR. In other embodiments, the target protein associated with Graves' disease is TSHR.

In some embodiments, the target protein comprises a protein selected from the group consisting of TNFα, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2 Frizzled receptors, Wnt, LRP5/6, CSF-1R, SIRPα, CD38, CD73, TGF-β, TSHRα, AChR-α1, noncollagen domain 1 of the α3 chain of type IV collagen (α3NC1), ADAMTS13, Desmoglein-1/3, GPIb/IX, GPIIb/IIIa, GPIa/IIa, NMDA receptor, glutamic acid decarboxylase (GAD), amphiphysin and gangliosides GM1, GD3, and GQ1B. In other embodiments, the target protein comprises an antibody that binds to TSHRα, MOG, AChR-α1, noncollagen domain 1 of the α3 chain of type IV collagen (α3NC1), ADAMTS13, Desmoglein-1/3, GPIb/IX, GPIIb/IIIa, GPIa/IIa, NMDA receptor, glutamic acid decarboxylase (GAD), amphiphysin and gangliosides GM1, GD3, and GQ1B.

In some embodiments, a method of delivering a target protein to a hepatocyte endosome is provided herein. In some embodiments, the method of delivering a target protein to a hepatocyte comprises contacting the target protein with any of the glycoengineered bifunctional binding proteins disclosed herein under conditions to mediate endocytosis of any of the target proteins disclosed herein. In some embodiments, the method of delivering the target protein to a hepatocyte endosome occurs in vivo. In some embodiments, the mode of delivering a target protein to a hepatocyte endosome in vivo comprises intravenous injection, intraperitoneal injection, subcutaneous injection, transdermal injection or intramuscular injection. In some embodiments, the method of delivering the target protein to a hepatocyte endosome occurs ex vivo.

In some embodiments, the rate of delivery can be increased based on the number of glycan structures present on the glycoengineered bifunctional binding protein. In some embodiments, increasing the number of glycan structures on the glycoengineered bifunctional binding protein increases the rate of delivery. In some embodiments, the glycoengineered bifunctional binding protein can comprise 1 or more, 2 or more, 3 or more, 4 or more, 5 or more, 6 or more, 7 or more, 8 or more, 9 or more or 10 or more glycan structures. In some embodiments, the glycan structure comprises GlcNAc2Man3GlcNAc2, GalNAc2GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan. In some embodiments, the glycan structure comprises GlcNAc2Man3GlcNAc2, GalNAc2GlcNAc2Man3GlcNAc2 or Gal2GlcNAc2Man3GlcNAc2. In some embodiments, the glycan structure is GlcNAc2Man3GlcNAc2. In some embodiments, the glycan structure is GalNAc2GlcNAc2Man3GlcNAc2. In some embodiments, the glycan structure is Gal2GlcNAc2Man3GlcNAc2.

In some embodiments, a method of degrading a target protein is provided herein. In some embodiments, the method of degrading a target protein comprises contacting the target protein with any of the glycoengineered bifunctional binding proteins disclosed herein under conditions to mediate degradation of any of the target proteins disclosed herein by a host cell. In some embodiments, degradation is lysosomal degradation. In some embodiments, degradation is mediated by endocytosis or phagocytosis. In some embodiments, degradation is at least 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than degradation mediated by a glycoengineered bifunctional binding protein comprising a glycan other than any of the second moieties disclosed herein. In other embodiments, the glycoengineered bifunctional binding protein enhances degradation of any of the disclosed target proteins relative to degradation of the target protein in the presence of a glycoengineered bifunctional binding protein comprising a glycan other than any of the second moieties disclosed herein. In some embodiments, the host cell is any host cell, including, but not limited to, a myeloid cell, an immune cell, an endothelial cell, a parenchymal cell or an epithelial cell. In some embodiments, the immune cell can be a dendritic cell, a macrophage, a monocyte, a microglia cell, a granulocyte or a B lymphocyte.

In some embodiments, the rate of lysosomal degradation can be regulated through glycoengineering. In some embodiments, the rate of lysosomal degradation can be regulated based on the number of glycan structures present on the glycoengineered bifunctional binding protein. In some embodiments, the rate of lysosomal degradation can be increased based on the number of glycan structures present on the glycoengineered bifunctional binding protein. In some embodiments, increasing the number of glycan structures on the glycoengineered bifunctional binding protein increases the rate of lysosomal degradation. In some embodiments, the glycoengineered bifunctional binding protein can comprise 1 or more, 2 or more, 3 or more, 4 or more, 5 or more, 6 or more, 7 or more, 8 or more, 9 or more or 10 or more glycan structures.

In some embodiments, the glycan structure comprises GlcNAc2Man3GlcNAc2, GalNAc2GlcNAc2Man3GlcNAc2, Gal2GlcNAc2Man3GlcNAc2, Man3GlcNAc, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan. In some embodiments, the glycan structure comprises GlcNAc2Man3GlcNAc2, GalNAc2GlcNAc2Man3GlcNAc2 or Gal2GlcNAc2Man3GlcNAc2. In some embodiments, the glycan structure is GlcNAc2Man3GlcNAc2. In some embodiments, the glycan structure is GalNAc2GlcNAc2Man3GlcNAc2. In some embodiments, the glycan structure is Gal2GlcNAc2Man3GlcNAc2. In some embodiments, the presence of two or more GlcNAc2Man3GlcNAc2 structures on the glycoengineered bifunctional binding protein can increase the rate of lysosomal degradation relative to a glycoengineered bifunctional binding protein comprising one GlcNAc2Man3GlcNAc2 structure. In some embodiments, the rate of lysosomal degradation can be fine-tuned. That is, the rate of lysosomal degradation can be increased by increasing the number of glycan structures present. Depending on the condition to be treated, different internalization rates are desired. For the treatment of an acute condition, rapid internalization of the complex between a bifunctional protein provided herein bound to its target protein(s) would be desired. To accomplish that at least 1, 2, 3, 4, 5, 6, 7, 8, 9, or 10 glycosites can be introduced and linked to N-glycans, which in turn results in a rapid internalization and low half lifes of the target protein of less than 10 minutes, 15 minutes, 30 minutes, 45 minutes, 60 minutes, 90 minutes, two hours, three hours, or less than four hours. For the treatment of an acute condition associated with increased levels of a target protein, the method comprises administering to a patient in need thereof a bifunctional binding protein, wherein the bifunctional binding protein (i) specifically binds to the target protein and (ii) comprises an N-glycan, wherein the N-glycan is linked to the bifunctional binding protein at one, two or more glycosites such that the half-life is at most 20%, 30%, 40%, 50%, 60%, 70%, 80%, 90% or 99% of the half-life of the target protein in the patient in the absence of the bifunctional binding protein or in the absence of any treatment.

For the treatment of a chronic condition, higher half lifes of the bifunctional protein provided herein would be desired. To accomplish that at most 1, 2, or at most 3, glycosites are present and linked to N-glycans, resulting in half lifes of more than 12 hours, 24 hours, 1, 2, 3, 4, 5, 6, 7, 8, 9, 10, 11, 12, 13, or 14 days, or more than 3 weeks or more than 4 weeks. For the treatment of a chronic condition associated with increased levels of a target protein, the method comprises administering to a patient in need thereof a bifunctional binding protein, wherein the bifunctional binding protein (i) specifically binds to the target protein and (ii) comprises an N-glycan, wherein the N-glycan is linked to the bifunctional binding protein at one or more glycosites, such that the half-life is at most 20%, 30%, 40%, 50%, 60%, 70%, 80%, 90% or 99% of the half-life of the target protein in the patient in the absence of the bifunctional binding protein or in the absence of any treatment.

In some embodiments, a method of degrading a target protein comprises GlcNAc mediated degradation. In some embodiments, GlcNAc degradation is optimal due to engagement of endocytic receptors. In some embodiments, the method of degrading a target protein via GlcNAc mediated degradation is selective. In some embodiments, GlcNAc degradation removes inflammatory cytokines from circulation, removes unwanted blood factors, removes autoantibodies and removes cell surface receptors.

In some embodiments, a method of degrading a target protein comprises GalNAc mediated degradation. In some embodiments, GalNAc degradation is optimal due to engagement of endocytic receptors. In some embodiments, the method of degrading a target protein via GalNAc mediated degradation is selective. In some embodiments, GalNAc degradation removes inflammatory cytokines from circulation, removes unwanted blood factors, removes autoantibodies, removes cell surface receptors and removes extracellular soluble proteins.

In some embodiments, a method of degrading a target protein comprises Gal mediated degradation. In some embodiments, Gal degradation is optimal due to engagement of endocytic receptors. In some embodiments, the method of degrading a target protein via Gal mediated degradation is selective. In some embodiments, Gal degradation removes inflammatory cytokines from circulation, removes unwanted blood factors, removes autoantibodies and removes cell surface receptors.

In some embodiments, a method of degrading a target protein comprises mannose 3 mediated degradation. In some embodiments, mannose 3 degradation is optimal due to engagement of endocytic receptors. In some embodiments, the method of degrading a target protein via mannose 3 mediated degradation is selective. In some embodiments, mannose 3 degradation removes inflammatory cytokines from circulation, removes unwanted blood factors, removes autoantibodies and removes cell surface receptors.

In some embodiments, the glycoengineered bifunctional binding protein that mediates degradation of a target protein is an anti-TGF-β monoclonal antibody comprising a Man3GlcNAc2 that captures TGF-β and degrades TGF-β in the lysosome via recognition of the Man3GlcNAc2 by any of the endocytic receptors disclosed herein. This approach can be applied to deplete a cell surface a receptor such Notch for cancer treatment.

In some embodiments, the target protein is a protein that is upregulated in cancer. In some embodiments, the target protein is a protein that is involved in cancer progression. Examples of target proteins that are upregulated in cancer or involved in cancer progression that can be bound by a glycoengineered bifunctional binding protein provided herein include, but are not limited to TNFα, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2 Frizzled receptors, Wnt, LRP5/6, CSF-1R, SIRPα, CD38, CD73, TGF-β, Bombesin R, CAIX, CD13, CD44v6, Emmprin, Endoglin, EpCAM, EphA2, FAP-α, Folate R, GRP78, IGF-1R, Matriptase, Mesothelin, sMET/HGFR, MT1-MMP, MT6-MMP, PSCA, PSMA, Tn antigen, and uPAR.

In some embodiments, the target protein is an autoantibody, such as those associated with an autoimmune disease. Examples of an autoantibody that can be bound by a glycoengineered bifunctional binding protein provided herein include, but are not limited to, autoantibodies directed against TSHRα, MOG, AChR-α1, noncollagen domain 1 of the α3 chain of type IV collagen (a3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B.

In some embodiments, the target protein comprises a protein that is upregulated or expressed in tumor associated macrophages (TAMs). In some embodiments, the target protein is upregulated or expressed in pro-tumor TAMs. Examples of target proteins that are upregulated or expressed in TAMs comprise SIRPα, CCR2, CSF-1R, LILRB1, LILRB2, VEGF-R, or CXCR4 (9). In other embodiments, the target proteins comprise CCL2, CXCL12, CSF-1 or CD47 (9). These targets, as described in reference (9), play a role in promoting pro-tumor TAMs particularly by promoting TAM recruitment and programming.

In some embodiments, provided herein is a method of reprogramming TAMs using any of the bifunctional binding proteins disclosed herein. As described in reference (7), reprogramming TAMs comprises targeting and inhibiting macrophage receptors to reprogram pro-tumorigenic TAMs into anti-tumorigenic TAMs. In other embodiments, provided herein is a method of depleting TAMs using any of the bifunctional binding proteins disclosed herein. Depletion of TAMs can occur by targeting receptors important for proliferation. Targeting of these receptors can promote apoptosis of pro-tumorigenic TAMs. In further embodiments, provided herein is a method of inhibiting recruitment of TAMs to the tumor microenvironment, using any of the bifunctional binding proteins disclosed herein. In some embodiments, the method of reprogramming, depleting or inhibiting recruitment of TAMs comprises using any of the disclosed bifunctional binding proteins to degrade a target upregulated or expressed by TAMs. In some embodiments, the target protein upregulated or expressed in TAMs comprises SIRPα, CCR2, CSF-1R, LILRB1, LILRB2, VEGF-R, or CXCR4. In other embodiments, the target protein associated with TAMs comprises CCL2, CXCL12, CSF-1 or CD47.

In some embodiments, provided herein is a pharmaceutical composition comprising the glycoengineered bifunctional binding protein described herein and a pharmaceutically acceptable carrier.

In some embodiments, provided herein is a method of treating or preventing a disease in a patient comprising administering to the patient a glycoengineered bifunctional binding protein described herein or a pharmaceutical composition described herein. In some embodiments, the disease is an autoimmune disease, a cancer or tumor, a liver disease, an inflammatory disorder, or a blood disorder. In some embodiments, the autoimmune disease is selected from Graves' Disease, Myasthenia Gravis, Anti-GBM Disease, Immune Thrombotic Thrombocytopenia Purpura, Acquired Pemphigus Vulgaris, Immune Thrombocytopenia, Guillain-Barré Syndrome, and Membranous Nephropathy. In some embodiments, the cancer or tumor is selected from breast cancer, colorectal cancer, pancreatic cancer, non-small cell lung cancer, hepatocellular carcinoma, and hematological T cell and B cell malignancies.

In some embodiments, a method of treating or preventing a disease provided herein includes an administration step that comprises intravenous injection, intraperitoneal injection, subcutaneous injection, transdermal injection, or intramuscular injection of a glycoengineered bifunctional binding protein described herein or a pharmaceutical composition described herein.

In some embodiments, a method of treating or preventing a disease provided herein requires a lower dose and/or lower administration frequency to achieve the same effect as compared to the same antibody having a different glycosylation profile; and/or can be administered for an extended period of time (at least 1, 2, 3, 4, 5, 6, 7, 8, 9, 10, 11 or at least 12 months, at least 2, 3, 4, 5, 6, 7, 8, 9, or at least 10 years); and/or does not trigger an immune response against the glycoengineered bifunctional binding protein in the patient.

In some embodiments, provided herein is a kit comprising the glycoengineered bifunctional binding protein of the present disclosure is provided herein. In some embodiments, the kit further provides instructions for administering the bifunctional molecule or pharmaceutical composition to an individual in need thereof.

In some embodiments, the pharmaceutical compositions described herein can be administered in a single dosage form, for example a single dosage form of a glycoengineered bifunctional binding protein described here.

In some embodiments, a suitable dose of a glycoengineered bifunctional binding protein described herein is the amount corresponding to the lowest dose effective to produce a therapeutic effect. For example, an effective amount of an anti-TSH receptor antibody can be an amount that inhibits TSH activity in a subject suffering from a Graves' disease.

In some embodiments, the amount of glycoengineered bifunctional binding protein described herein administered to a patient is less than the amount listed in the label of a drug product of the same glycoengineered bifunctional binding protein having a different glycosylation profile from that of the glycoengineered bifunctional binding protein described herein.

In some embodiments, the accumulated amount of a glycoengineered bifunctional binding protein described herein administered to a patient over a period of time is less than the accumulated amount indicated in the label of a drug product of the same glycoengineered bifunctional binding protein having a different glycosylation profile from that of the glycoengineered bifunctional binding protein described herein. In some embodiments, the reduced accumulated amount could be administered in reduced doses on a reduced frequency. In some embodiments, the reduced accumulated amount could be administered in one or more doses that are the same or higher than the dose in the label on a reduced frequency. In some embodiments, the reduced accumulated amount could be administered in one or more reduced doses on a frequency that is the same or higher than the frequency in the label. In some embodiments, the reduced accumulated amount could be administered over a shorter period of time than the period of time for the drug product to achieve the same level of effect in treatment or prevention.

In some embodiments, the amount of the glycoengineered bifunctional binding protein described herein in a single dose administered to a patient can be from about 1 to 150 mg, about 5 to 145 mg, about 10 to 140 mg, about 15 to 135 mg, about 20 to 130 mg, about 25 to 125 mg, about 30 to 120 mg, about 35 to 115 mg, about 40 to 110 mg, about 45 to 105 mg, about 50 to 100 mg, about 55 to 95 mg, about 60 to 90 mg, about 65 to 5 mg, about 70 to 80 mg, or about 75 mg. In some embodiments, the amount of glycoengineered bifunctional binding protein described herein in a single dose administered to a patient can be from about 5 to about 80 mg. In some embodiments, the amount of glycoengineered bifunctional binding protein described herein in a single dose administered to a patient can be from about 25 to about 50 mg. In some embodiments, the amount of a glycoengineered bifunctional binding protein described herein in a single dose administered to a patient can from about 15 mg to about 35 mg.

In some embodiments, the amount of a glycoengineered bifunctional binding protein described herein in a single dose administered to a patient can be no more than 40 mg, for example 40 mg, 35 mg, 30 mg, 25 mg, 20 mg, 18 mg, 15 mg, 12 mg, 10 mg, 7 mg, 5 mg, and 2 mg. In some embodiments, the amount of a glycoengineered bifunctional binding protein described herein in a single dose administered to a patient can be no more than 80 mg, for example 80 mg, 75 mg, 70 mg, 65 mg, 60 mg, 55 mg, 50 mg, 45 mg, 40 mg, 35 mg, 30 mg, 20 mg, 15 mg, 10 mg, 5 mg and 2 mg. In some embodiments, the amount of a glycoengineered bifunctional binding protein described herein in a single dose administered to a patient can be no more than 160 mg, for example 150 mg, 140 mg, 130 mg, 120 mg, 110 mg, 100 mg, 90 mg, 80 mg, 75 mg, 70 mg, 65 mg, 60 mg, 55 mg, 50 mg, 45 mg, 40 mg, 35 mg, 30 mg, 20 mg, 15 mg, 10 mg, 5 mg and 2 mg. In some embodiments, the amount of a glycoengineered bifunctional binding protein described herein in a single dose administered to a patient can be equal to or more than 160 mg, for example 170 mg, 180 mg, 200 mg, 250 mg, and 300 mg.

In some embodiments, a glycoengineered bifunctional binding protein of the disclosure can be administered on a frequency that is every other week, namely every 14 days. In some embodiments, a glycoengineered bifunctional binding protein of the disclosure can be administered on a frequency that is lower than every 14 days, for example, every half a month, every 21 days, monthly, every 8 weeks, bimonthly, every 12 weeks, every 3 months, every 4 months, every 5 months, or every 6 months. In some embodiments, a glycoengineered bifunctional binding protein of the disclosure can be administered on a frequency that is the same or higher than every 14 days, for example, every 14 days, every 10 days, every 7 days, every 5 days, every other day, or daily.

In some embodiments, the administration of a glycoengineered bifunctional binding protein of the disclosure can comprise an induction dose that is higher than the following doses, for example the following maintenance doses. In some embodiments, the administration of a glycoengineered bifunctional binding protein of the disclosure can comprise a second dose that is lower than the induction dose and higher than the following maintenance doses. In some embodiments, the administration of a glycoengineered bifunctional binding protein of the disclosure can comprise the same amount of the glycoengineered bifunctional binding protein in all the doses throughout the treatment period.

Methods of generating a glycoengineered bifunctional binding protein provided herein are well known in the art. Exemplary methods of generating a glycoengineered bifunctional binding protein provided herein are described in International Patent Application Publications WO 2019/002512, WO 2021/140143, WO 2021/140144, and WO 2022/053673, which are incorporated herein by reference in their entirety, and are exemplified herein, any one of which can be used to generate a glycoengineered bifunctional binding protein provided herein. For example, one of skill in the art will readily appreciate that the nucleic acid sequence of a known protein (e.g., a monoclonal antibody), as well as a newly identified protein (e.g., a monoclonal antibody), can easily be deduced using methods known in the art, and thus it would be well within the capacity of one of skill in the art to introduce a nucleic acid that encodes any glycoengineered bifunctional binding protein into a host cell provided herein (e.g., via an expression vector, e.g., a plasmid, e.g., a site specific integration by homologous recombination).

In some embodiments, provided herein is a Leishmania host cell comprising the glycoengineered bifunctional binding protein described herein. Such a host cell, in some embodiments, is Leishmania tarentolae. In some embodiments, the host cell is a Leishmania aethiopica cell. In some embodiments, the host cell is part of the Leishmania aethiopica species complex. In some embodiments, the host cell is a Leishmania aristidesi cell. In some embodiments, the host cell is a Leishmania deanei cell. In some embodiments, the host cell is part of the Leishmania donovani species complex. In some embodiments, the host cell is a Leishmania donovani cell. In some embodiments, the host cell is a Leishmania chagasi cell. In some embodiments, the host cell is a Leishmania infantum cell. In some embodiments, the host cell is a Leishmania hertigi cell. In some embodiments, the host cell is part of the Leishmania major species complex. In some embodiments, the host cell is a Leishmania major cell. In some embodiments, the host cell is a Leishmania martiniquensis cell. In some embodiments, the host cell is part of the Leishmania mexicana species complex. In some embodiments, the host cell is a Leishmania mexicana cell. In some embodiments, the host cell is a Leishmania pifanoi cell. In some embodiments, the host cell is part of the Leishmania tropica species complex. In some embodiments, the host cell is a Leishmania tropica cell.

In some embodiments, provided herein is a method for making a glycoengineered bifunctional binding protein comprising culturing a Leishmania host cell described herein and isolating the glycoengineered bifunctional binding protein.

In some embodiments, provided herein is a glycoengineered bifunctional binding protein produced by the method described herein.

Methods of producing a Leishmania host cell and using such host cells to produce a glycoengineered bifunctional binding protein are well known in the art. Exemplary methods are described in International Patent Application Publications WO 2019/002512, WO 2021/140143, WO 2021/140144 and WO 2022/053673, which are incorporated herein by reference in their entirety, and are exemplified herein, any one of which can be used to generate a Leishmania host cell and produce a glycoengineered bifunctional binding protein provided here. For example, in some embodiments, host cells described herein are cultured using any of the standard culturing techniques known in the art, including, but not limited to, growth in rich media like Brain Heart Infusion, Trypticase Soy Broth or Yeast Extract, all containing 5 μg/ml Hemin. Additionally, incubation can be done at 26° C. in the dark as static or shaking cultures for 2-3 days. In some embodiments, cultures of host cell contain the appropriate selective agents. For example, in some embodiments, monoclonal antibody described herein is purified from host cell culture supernatants using any of the standard purification techniques known in the art, including, but not limited to, Protein A Affinity Chromatography, Ion Exchange Chromatography and Mixed Mode Chromatography.

Other methods to generate the bifunctional proteins provided herein can be used. For example, chemical conjugation or chemo-enzymatic modifications can be used to generate the bifunctional proteins provided herein

In some embodiments, provided herein is a method of treating an acute condition associated with increased levels of a target protein, wherein the method comprises administering to a patient in need thereof a bifunctional binding protein, wherein the bifunctional binding protein (i) specifically binds to the target protein and (ii) comprises an N-glycan of the structure:

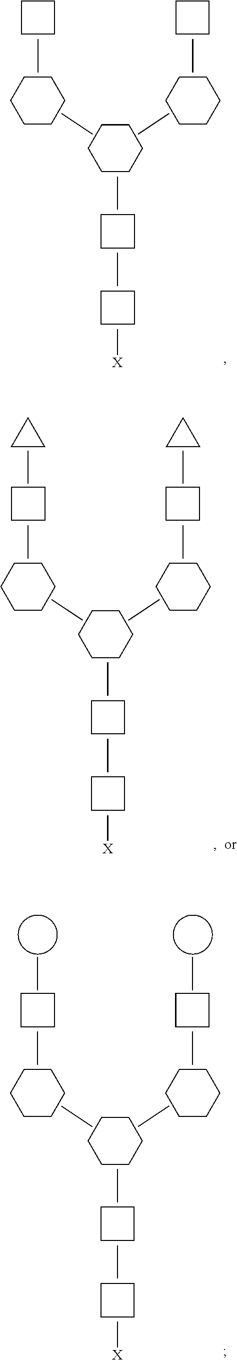

wherein the N-glycan is linked to the bifunctional binding protein at at least 1, 2, 3, 4 or 5 N-glycosylation sites (symbols have the meaning introduced above), which results in half-life that is at least 50%, 60%, 70%, 80%, 90% or 99% of the bifunctional binding protein without any glycosylation. In some embodiments, the half-life of the target protein in the present of a bifunction protein provided herein in a patient is 5 minutes, 10 minutes, 15 minutes, 30 minutes, 45 minutes, 1 hour, 1.5 hours, 2 hours, 2.5 hours, or 3 hours.

In some embodiments, provided herein is a method of treating a chronic condition associated with increased levels of a target protein, wherein the method comprises administering to a patient in need thereof a bifunctional binding protein, wherein the bifunctional binding protein (i) specifically binds to the target protein and (ii) comprises an N-glycan of the structure:

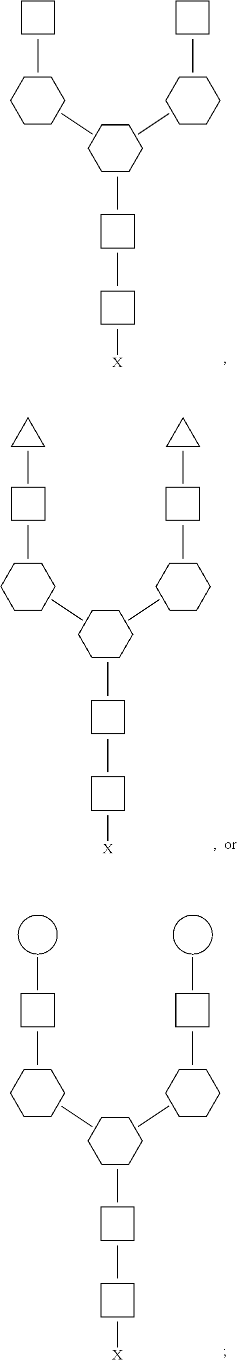

wherein the N-glycan is linked to the bifunctional binding protein at at most 1, 2, 3, 4 or 5 N-glycosylation sites (symbols have the meaning introduced above), which results in a half-life that is at least 50%, 60%, 70%, 80%, 90% or 99% of the bifunctional binding protein without any glycosylation in the patient. In some embodiments, the half-life of the target protein is at least 6 hours, 12 hours, 18 hours, 1 day, 2 days, 3 days, 4 days, 5 days, 6 days or 7 days.

In some embodiments, provided herein is a method of treating a chronic condition associated with increased levels of a target protein, wherein the method comprises administering to a patient in need thereof a bifunctional binding protein, wherein the bifunctional binding protein (i) specifically binds to the target protein and (ii) comprises an N-glycan, wherein the N-glycan is linked to the bifunctional binding protein at one, two or more glycosites such that the half-life is at most 20%, 30%, 40%, 50%, 60%, 70%, 80%, 90% or 99% of the half-life of the target protein in the patient in the absence of the bifunctional binding protein or in the absence of any treatment.

In some embodiments, the chronic condition is an autoimmune disease, a cancer or tumor, a liver disease, an inflammatory disorder, or a blood coagulation disorder.

In some embodiments, the autoimmune disease is selected from Graves' Disease, Myasthenia Gravis, Anti-GBM Disease, Immune Thrombotic Thrombocytopeniaurpura, Acquired Pemphigus Vulgaris, Immune Thrombocytopenia, autoimmune encephalitis, Guillain-Barré Syndrome, and Membranous Nephropathy.

In some embodiments, the cancer comprises a solid tumor. In some embodiments, the cancer comprises a blood-borne cancer or tumor. In some embodiments, the cancer may be a carcinoma or a sarcoma. In some embodiments, the cancer is selected from lung cancer (small cell or non-small cell), breast cancer, gastric cancer, colorectal cancer, bladder cancer, malignant melanoma, brain cancer (e.g., astrocytoma, glioma, meningioma, neuroblastoma, or others), bone cancer (e.g., osteosarcoma), cervical cancer, cholangiocarcinoma, digestive tract cancer (e.g., oral, esophageal, stomach, colon or rectal cancer), head and neck cancer, leiomyosarcoma, liposarcoma, liver cancer (e.g., hepatocellular carcinoma), mesothelioma, nasopharyngeal cancer, neuroendocrine cancer, ovarian cancer, pancreatic cancer, prostate cancer, renal cancer, rhabdomyosarcoma, salivary gland cancer, skin cancer, spindle cell carcinoma, testicular cancer, thyroid cancer, or uterine cancer (e.g., endometrial cancer). In certain embodiments, the cancer can be relapsed following a previous therapy, or refractory to conventional therapy. In certain embodiments, the cancer can be disseminated or metastatic. In some embodiments, the blood-borne cancer or tumor is selected from leukemia, myeloma (e.g., multiple myeloma) lymphoma (e.g., Hodgkin's lymphoma or non-Hodgkin's lymphoma). In certain embodiments, the leukemia is chronic lymphocytic leukemia, chronic myeloid leukemia, acute lymphocytic leukemia, acute myelogenous leukemia and acute myeloblastic leukemia. In some embodiments, treatment comprises reprogramming tumor associated macrophages (TAMs) by administering the bifunctional binding protein under conditions to mediate endocytosis of a target protein.

In some embodiments, the target protein is upregulated or expressed in TAMs.

In some embodiments, the target protein upregulated or expressed in TAMs comprises SIRPα, CCR2, CSF-1R, LILRB1, LILRB2, VEGF-R, CXCR4, CCL2, CXCL12, CSF-1 or CD47.

In some embodiments, the administration step comprises intravenous injection, intraperitoneal injection, subcutaneous injection, transdermal injection, or intramuscular injection.

It is understood that modifications which do not substantially affect the activity of the various embodiments of this invention are also provided within the definition of the invention provided herein. Accordingly, the following examples are intended to illustrate but not limit the present invention.

### Example I

### Generation of Glycosylated Bifunctional Antibodies.

Antibodies were prepared as follows. The monoclonal anti-TNF alpha antibodies, HUMIRA® (Abb Vie) or Leishmania tarentolae CGP derived adalimumab variants (A-S, A-M), or Mabthera were re-buffered to 30 mM MES buffer pH 6.5 using ZebaSpin columns (ThermoFischer, US). The galactosylation and alpha 2,6-sialylation (to generate H-S and A-S variants) was performed using in vitro glycosylation (IVGE, Roche Diagnostics) in an 1-pot reaction at 37° C. under mild rotation according to the application note. The glycosylated mAb was purified from the reaction mixture with ProteinA sepharose (MabSelectSuRe or HiTrap MabSelect PrismA column GE Healthcare) according to manufacturer's recommendation using FPLC (Bio-Rad NGC, Germany). Thereafter, a desalting procedure using PD-10 (Sephadex 25, Sigma, Switzerland) was carried out for a buffer exchange to PBS pH 6 (137 mM NaCl, 2.7 mM KCl, 8.6 mM NaH2PO4, 1.4 mM Na2HPO4, Sigma, Switzerland) followed by sterile filtration using 0.2 μm PES filter (ThermoFisher, US). Quality and glycosylation of antibodies (Table 2) was analyzed using GlycoWorks RapiFluor-MS N-Glycan Kit (Waters, US) according to the manufacturer's instructions or by procainamide (PC) labelling of PNGaseF released glycans. For additional N-glycan analysis, the monoclonal antibody was either cleaved with IdeZ to F(ab′)2 and Fc/2 (For IgG1); or alternatively reduced to heavy and light chains (for IgG4), separated on SDS PAGE and bands were excised and enzymatic release of N-glycans from the monoclonal antibody was performed using PNGase F. Following release, glycans were labeled with procainamide (PC). PC-labeled N-glycans were analyzed by HILIC-UPLC-MS with fluorescence detection coupled to a mass spectrometer. Glycans were separated using an Acquity BEH Amide column. Data processing and analysis was performed using Unifi. Glucose units were assigned on the retention times of a procainamide-labeled dextran ladder. Glycan structures were assigned based on their m/z values and their retention times. Glycan forms and relative percentages were calculated based on peak areas. SE-HPLC analysis was performed at a concentration of 1 μg/μL on a MabPac (ThermoFischer, US) column and run according to the manufacturer's instructions and endotoxin levels were below 0.2 EU/mg (Endosafe®). Table 2 shows the main N-glycan structure displayed by the antibodies used.

TABLE 2
Main glycoforms of pHrodo-labeled antibodies.

| | | Glycan | |
|---|---|---|---|
| Antibody | Abbreviation | nomenclature | Main N-glycan structure |
| Mabthera (rituximab) | MbT | A2F | 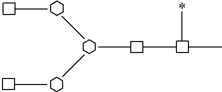 |
| HUMIRA ® (adalimumab) | H-M | A2F | 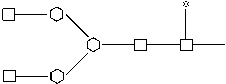 |
| Man3-adalimumab | A-M | Man3 | 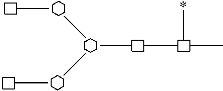 |
| Sialylated- adalimumab | A-S | A2G2S2 | 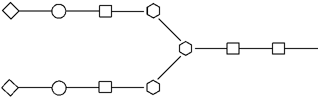 |
| Sialylated- HUMIRA ® | H-S | A2G2S2F | 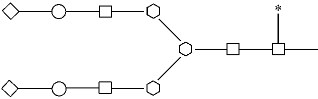 |

Table 2 shows the main N-glycoform (canonical N-297 position) displayed by indicated antibodies. The short IgG nomenclature is typically used with IgG associated naming system. It indicates the presence of core fucose, the number of galactoses and the presence of biantennary glycans. It is limited in the number of structures and linkages it can describe but is often used for simplicity. Hexagon represents mannose (Man), white square is N-acetyl glucosamine (GlcNAc), white circle is galactose (Gal), white diamond is sialic acid, N-acetyl neuraminic acid (Neu5Ac), and asterisk is fucose (Fuc).

Antibodies were labeled with pHrodo dye (pHrodo iFL Red STP Ester [amine-reactive], ThermoFisher, ref. P36011) according to manufacturer instructions. The fluorescence of pHrodo is activated at low pH and therefore will allow for the visualization of protein internalization and targeting to the lysosomal pathway.

The pHrodoDegree of Labeling (DOL) for each antibody was determined as follows. Antibodies were diluted 1:2 in denaturing buffer and analyzed with Nanodrop at 280 nm and 560 nm wavelength (A280 and A560). Protein concentration and pHrodo DOL were calculated as follows.

$$Protein conc . [ M ] = \frac{protein conc . [ \frac{mg}{ml} ]}{MW [ g / mol ]} = \frac{protein conc . [ \frac{mg}{ml} ]}{144000}$$

$$DOL = \frac{{λ}_{m ax} * dilution factor}{{ε}_{dye} [ M - 1 cm - 1 ] * protein conc . [ M ]} = \frac{A 560 * 2}{65000 * protein conc . [ M ]}$$

MW is the molecular weight of the antibody used: 144000 g/Mol. λmax is the absorbance measured at 560 nm. εdye is the Extinction coefficient: 65000 M⁻¹ cm⁻¹. Dilution factor is 2.

Antibodies used in these experiments are shown in Table 3 along with their main N-glycan structure on Fc or Fab parts.

TABLE 3
Main N-glycan structure on antibodies.

| | | Engineered | | |
|---|---|---|---|---|
| | | Glycosites | Main N-glycan structure on | Main N-glycan structure on |
| Antibody | Abbreviation | (Eu numbering) | Fc | Fab |
| Mabthera (rituximab) | M-A2F | None | 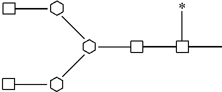 | No |
| HUMIRA ® (adalimumab) | H-A2F | None | 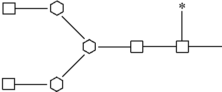 | No |
| Deglycosylated Humira | H-PNGase | None | No | No |
| Fc-Man3- adalimumab | A-M3 | None | 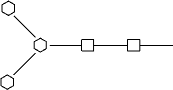 | No |
| Fab-Man3- adalimumab | A8486-M3 | LC: D86N HC: K84N | 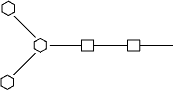 | 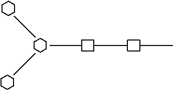 |
| Fab-Sia- adalimumab | A8486- A2G2S2 | LC: D86N HC: K84N | 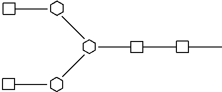 | 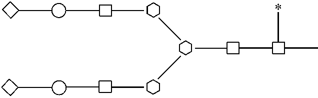 |

Main N-glycan structure on Fab means on the engineered glycosite by point mutations (Eu numbering). LC is light chain, HC is heavy chain. Hexagon
represents mannonse (Man), white square is N-acetyl glucosamine (GlcNAc), white circle is galactose (Gal), with diamond is sialic acid, N-acetyl
neuraminic acid (Neu5Ac), and asterisk is fucose (Fuc).

### Example II

### Galnac2 Glycosylated Fab Antibody Leads to Efficient Internalization Via ASGPR in Hepatocyte Cells

HepG2 hepatocarcinoma cells (ATCC #HB-8065) express ASGPR and were maintained in a low glucose DMEM medium (Sigma, Ref. D5546) supplemented with 10% FBS. Adalimumab antibodies (A-8486-A2; -A2G2; and -A2GalNAc2) were purified from CGP cell culture supernatant with Protein A HiTrap Mabselect PrismA, (Cytiva) and Capto™ adhere ImpRes (Cytiva) and formulated in PBS buffer pH 6.4, by using first Concentrator PES, 30K MWCO, 5-20 mL (15451025; Thermo Fisher), and PD-10 Desalting columns (Disposable PD-10 Desalting Column; 17085101; Cytiva) Antibodies were labeled with pHrodo as described in Example I. Table 4 shows the antibody characteristics that were included in the study. All antibodies showed aggregate levels below 5% as assessed by size exclusion HPLC method and higher than 90% purity as assessed by reducing polyacrylamide gel electrophoresis analysis and had an endotoxin level lower than 1.5 EU/mg (LAL assay).

TABLE 4
Antibody characteristics.

| | Engineered | | | |
|---|---|---|---|---|
| | Glycosites | | | |
| | (Eu | Main N-glycan | Main N-glycan structure on | |
| Antibody | numbering) | structure on N297 (Fc) | engineered glycosites | N-Glycan |
| H-A2F | None | 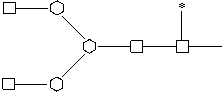 | NA | 71% A2F |
| A-8486-A2 | LC: D86N HC: K84N | 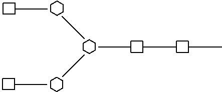 | 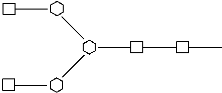 | Fab: 98% A2 Fc: 96% A2 |
| A-8486- A2G2 | LC: D86N HC: K84N | 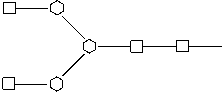 | 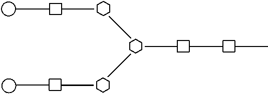 | Fab: 92% A2G2 Fc: 64% A2 |
| A-8486- A2GalNAc2 | LC: D86N HC: K84N | 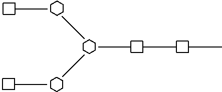 | 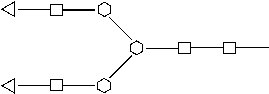 | Fab: 71% A2GalNAc2/15% A2GalNAc1 Fc: 95% A2 |

NA: not applicable. Main N-glycan structure on Fc is on the canonical N-297 position. Main N-glycan structure on Fab means on the
engineered glycosite by point mutations (Eu numbering). LC is light chain, HC is heavy chain. Hexagon represents mannose (Man), white
square is N-acetyl glucosamine (GlcNAc), white circle is galactose (Gal), triangle is N-acetyl galactosamine (GalNAc), and asterisk is
fucose (Fuc).

HepG2 cell monolayers were incubated for 3 to 4 hours or 24 hours with pHrodo-antibodies (3 μg/ml)+IVIg (1 mg/ml) (Hizentra, obtained from pharmacy), at 37° C. In some conditions, cells were also treated with the following reagents: fetuin (Sigma, Ref. F3385) at 2 μM; asialofetuin (Sigma, Ref. A4781) at 2 μM; Chloroquine (Sigma, Ref. C6628) at 50 μM; Bafilomycin (Millipore, Ref. 19148) at 10 nM; cytochalasin D (Sigma, Ref. C2618) at 50 μM. Cell cultures were then washed, and harvested using Accutase (Sigma/Merck, Ref. SCR005) and immediately acquired on a flow cytometer. Mean fluorescence intensity (MFI) of pHrodo for gated single and live (DAPI+ cells excluded) cell population was analyzed using standard flow cytometry software. MFI values were adjusted to pHrodo DOL.

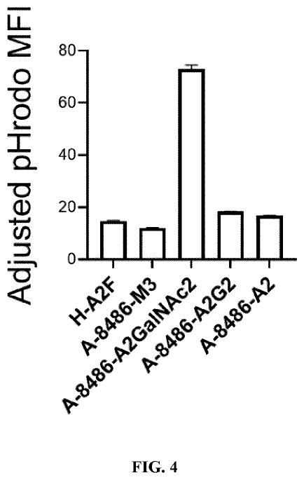

FIG. 4 shows the data obtained comparing H-A2F (Adalimumab; Humira, obtained from pharmacy), A-8486-A2, A-8486-A2G2, A-8486-A2GalNAc2 and A-8486-M3 antibodies. After 4 hours of incubation, only GalNAc2 displaying antibodies were internalized in HepG2 cells, indicating that GalNAc2 is a potent glycan for recognition and internalization by hepatocyte cells.

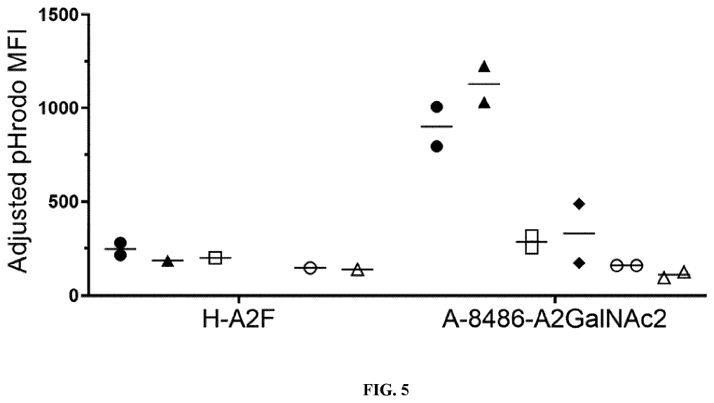

Another experiment to assess whether the uptake of GalNAc2 antibody is mediated by ASGPR was performed by using different inhibitors. FIG. 5 shows the data obtained in this inhibition experiment. The internalization of A-84.86-A2GalNAc2 was inhibited by EGTA, a calcium ion chelator, indicating that uptake of the antibody is likely mediated by a calcium-dependent C-type lectin receptor. In addition, internalization was selectively inhibited by asialofetuin (ligand for ASGPR) but not by fetuin (not a ligand for ASGPR), indicating that the recognition and internalization of GalNAc2-antibodies is mediated by ASGPR (Braun et al. J Biol Chem, 271 (35): 21160-6 (1996). The pHrodo signal produced by A-84.86-A2GalNAc2 was completely blunted by chloroquine and bafilomycin that disrupt clathrin-mediated endocytosis by blocking endosomal acidification (Ippoliti et al. Cell Mol Life Sci, 56:866-875 (1998). Endosomal acidification is a key step to achieve lysosomal protein degradation.

The pattern of inhibition observed in these experiments is consistent with an endocytic mechanism that is mediated by ASGPR, relies on clathrin, but not other endocytic pathways and that directs the endocytosed material to lysosomal compartment. Therefore these data support the use of CGP-produced GalNAc2 displaying proteins to target compounds to ASGPR-mediated internalization and degradation.

### Example III

### A2 Glycosylated Fab Antibody Leads to Potent In Vivo Depletion of a Blood Circulating Antigen

To assess the potency of antibodies produced in CGP and displaying GlcNAc terminated glycans (A2 structure) to deplete a circulating antigen, an experiment in rat was designed. Rats were injected with an antigen and with antibodies displaying A2 glycan structure on their Fab or control antibodies, specific for the antigen. The level of the circulating antigen in the serum of treated animals was quantified along time to measure the extend of depletion of the antigen from peripheral blood compartment. Table 5 shows the antibody characteristics that were included in the study. All antibodies showed aggregate levels below 5% as assessed by size exclusion HPLC method and higher than 94% purity as assessed by reducing polyacrylamide gel electrophoresis analysis and had an endotoxin level lower than 1 EU/mg (LAL assay). All glycoengineered antibodies conserved high binding to HCA202.

TABLE 5
Antibody characteristics

| | | | | Binding | |
|---|---|---|---|---|---|
| | | | | to | |
| | Engineered | | | HCA202 | |
| | Glycosites | Main N-glycan | | (% of H- | |
| | (Eu | structure of N297 | Main N-glycan structure on | A2F | |
| Antibody | numbering) | (Fc) | engineered glycosites | antibody) | N-Glycan |
| H-A2F | None | 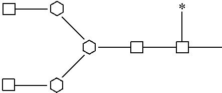 | NA | 100 | 71% A2F |
| A-M3 | None | 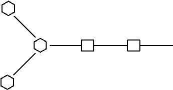 | NA | 87 | 98% M3 |
| A-84-82 | HC: K84N | 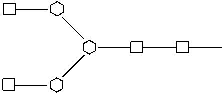 | 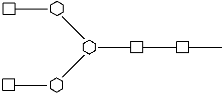 | 63 | Fab: 98% A2 Fc: 94% A2 |
| A-8486-A2 | LC: D86N HC: K84N | 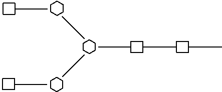 | 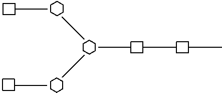 | 71 | Fab: 98% A2 Fc: 96% A2 |
| A-8486- A2G2S2 | LC: D86N HC: K84N | 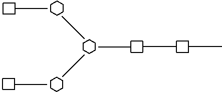 | 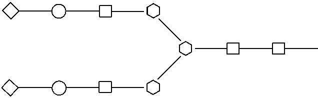 | 108 | Fab: 48% S2/ 27% S1 Fc: 52% A2/ 2% M3 |

NA: not applicable. Main N-glycan structure on Fc is on the canonical N-297 position. Main N-glycan structure on Fab means on the
engineered glycosite by point mutations (Eu numbering). LC is light chain, HC is heavy chain. Binding to HCA202 was performed by ELISA and
compared binding EC₅₀ values. Hexagon represents mannose (Man), white square is N-acetyl glucosamine (GlcNAc), white circle is galactose (Galtriangle
is N-acetyl galactosamine (GalNAc), white diamond is sialic acid, N-acetyl neuraminic acid (Neu5Ac), and asterisk is fucose (Fuc).

Wistar female rats (Janvier Labs, St Berthevin, France, ref. RjHan: WI) 180-220 g at start of experiment were injected i.v. bolus with anti-adalimumab Fab fragment HCA202 (Biorad, ref. HCA202) (the antigen) at 0.5 mg/kg dose, 0.5 ml/rat. HCA202 compound was submitted prior to injection to an endotoxin removal step using Pierce™ High Capacity Endotoxin Removal Spin Columns (Thermofisher, ref. 88274). Fifteen (15) min later rats were injected with antibodies (Table 4 and Table 5) or PBS. Blood samples were taken from jugular vein by puncture at following time points: 15 to 30 min after antibody injection, 1 hour, 6 hours and 24 hours. Terminal blood samples were collected at 48 hours from abdominal aorta. Blood samples were left for clotting 30 min at room temperature followed by centrifugation to collect serum.

Total HCA202 levels (antibody bound+free HCA202) were measured by ELISA method. Anti-Penta-His antibody (Qiagen, Ref. 34660) was coated on 96-well ELISA plates at 5 μg/ml in coating buffer (PBS pH 7.4, final composition: 8 mM Na-Phosphate; 8 mM K-Phosphate, 0.15 M NaCl, 10 mM KCl) overnight at 4° C. This antibody allows the capture of antibody bound and free HCA202 as HCA202 has an histidine tag at C terminus of heavy chain. Plates were washed 3 times with wash buffer (PBST=PBS with 0.05% v/v Tween-20). Blocking buffer (2% (w/v) Bovine serum albumin (BSA) in PBST) was added to each well and plates were incubated for 1-3 hours at room temperature. A 7-point calibration curve from 500 ng/ml to 0.7 ng/ml in 1:3 dilutions was made on separate dilution plates. For that, undiluted normal rat wistar serum was spiked with 5 μg/ml HCA202 and 3 fold molar excess of adalimumab (Humira). Spiked serum were incubated 10 min at room temperature to allow adalimumab/HCA202 immune complex formation. The spiked serum were diluted 10 fold (MRD10) by adding diluent B (2% (w/v) Bovine serum albumin (BSA) in PBST). A serial 1:3 dilution of the immune complex standard curve was performed using diluent B. Study samples were processed similarly. Study serum samples were diluted 10 fold in dilution plates (to achieve MRD10 samples) using diluent B. MRD10 samples were further diluted if needed in diluent A (1/10 wistar rat pooled serum diluted in 2% BSA+0.05% PBST) to achieve a signal within the linear range of the calibration standard curve. After blocking step, ELISA plates ware washed 3 times with wash buffer. Calibration standards and diluted study samples were added in duplicates to ELISA plates and incubated at room temperature for 1h. ELISA plates were washed 3 times with wash buffer and a Humira solution at 1000 ng/ml was added to each sample. ELISA plates were incubated for 1 h at room temperature. Plates were washed 3 times with wash buffer. A detection antibody solution was prepared by diluting goat anti-human kappa LC-HRP (Thermofisher, ref. A18853) 1:5000 in diluent B. The detection antibody solution was added to the ELISA plates and incubated for 1 h at room temperature, protected from light. ELISA plates were then washed 3 times with wash buffer and revealed by addition of TMB substrate followed by quenching with H₂SO₄. ELISA plates were read at 450 and 650 nM on a plate reader such as BioTek Synergy H1. Data analysis was made using standard software such as Gen5 (Biotek).

Antibody levels in serum sample can be quantified by ELISA method. The assay consists of a coating step with human TNFα to capture adalimumab and adalimumab variants present in the sample. Detection can be performed via an anti-human gamma HC specific HRP-tagged detection antibody. The assay therefore quantifies only free antibodies (having at least one Fab arm not bound to HCA202). Briefly, recombinant Human TNF-α (Peprotech, ref. AF-300-01A) is coated on 96-well ELISA plates, typically at 1 μg/ml in PBS pH 7.4 at 4° C. overnight. Blocking buffer, dilution buffer A and B and wash buffer are the same than used for the HCA202 ELISA. Plates are washed 3 times and blocked with blocking buffer as described in the HCA202 ELISA. Typically, a 7-point calibration curve, for example from 333.3 ng/ml to 0.5 ng/ml in 1:3 dilutions is prepared by spiking pooled wistar rat serum diluted 10 fold (minimal required 10 fold dilution, MRD10) in dilution buffer B with 1 μg/ml adalimumab. Study serum samples are also diluted in dilution buffer B (MRD10 samples minimum). Diluted study samples and standard calibration curve samples are then transferred to the ELISA plate, after blocking step and incubated 1 h at room temperature. Plates are then washed 3 times and solution of detection antibody is added. Solution of detection antibody can be prepared by diluting for example a Goat Anti-Human IgG (γ-chain specific)-HRP (Sigma, ref. A6029) antibody (typical dilution 1:10′000) in dilution buffer B. ELISA plates are incubated with detection antibody typically 1h at room temperature, protected from light. Plates are then washed 3 times and revealed by adding TMB substrate as described in the HCA202 ELISA.

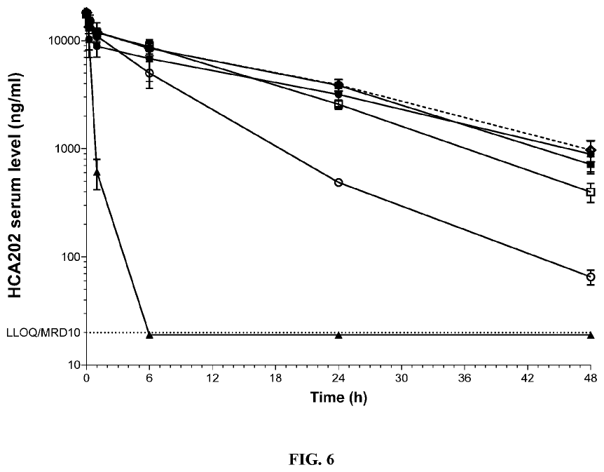

FIG. 6 shows the data obtained for HCA202 levels. When no antibody is injected (PBS condition), HCA202 decays slowly over period of 48h, as expected for a Fab fragment. H-A2F (non-engineered adalimumab) treatment led to increase levels of HCA202 at 24 and 48 hours time point. Similarly treatment with A-M3, A-84-A2 and A-8486-A2G2S2 led to increased HCA202 levels as compared to PBS treatment (72% depletion from Czero with PBS vs 52-63% depletion with H-A2F, A-M3 and A-8486-A2G2S2 at 6h). Czero is the theoretical concentration (of HCA) in serum that would have been achieved immediately post injection, considering immediate homogeneous whole blood distribution. In contrast injection of A-8486-A2 led to a drastic decrease of HCA202 levels already at early time points (97% depletion at 1h). Table 6 shows the HCA202 depletion numbers

TABLE 6
HCA202 depletion by A2 glycosylated Fab antibodies

| | | | | A-8486- | | |
|---|---|---|---|---|---|---|
| | PBS | H-A2F | A-M3 | A2G2S2 | A-84-A2 | A-8486-A2 |
| % depletion at | 39 | 51 | 34 | 31 | 33 | 97 |
| 1 h | | | | | | |
| % depletion at | 72 | 63 | 53 | 52 | 50 | 100 |
| 6 h | | | | | | |

Table 6 shows the % of HCA202 depletion from Czero.

These data highlight that antibodies displaying A2 glycans on their Fab fragment have a high potency to eliminate a circulating antigen from blood circulation in a very short time. These data also show that the glycan load displayed per molecule is critical to enable the depletion potency, as only the A-8486-A2 antibody with two engineered glycosites per Fab showed depletion, while A-84-A2 antibody with a single glycosite per Fab was non-depleting.

### Example IV

### A2G2 Glycosylated Fab Antibody Leads to Potent In Vivo Depletion of a Blood Circulating Antigen

To assess the potency of antibodies produced in CGP and displaying Galactose terminated glycans (A2G2 structure) to deplete a circulating antigen, an experiment in rat was designed. Rats were injected with an antigen and with antibodies displaying A2G2 glycan structure on their Fab or control antibodies, specific for the antigen. The level of the circulating antigen in the serum of treated animals was quantified along time to measure the extend of depletion of the antigen from peripheral blood compartment. Table 7 shows the antibody characteristics that were included in the study. All antibodies showed aggregate levels below 5% as assessed by size exclusion HPLC method and higher than 94% purity as assessed by reducing polyacrylamide gel electrophoresis analysis and had an endotoxin level lower than 1 EU/mg (LAL assay). All glycoengineered antibodies conserved high binding to HCA202.

TABLE 7
Antibody characteristics

| | | | | Binding | |
|---|---|---|---|---|---|
| | | | | to | |
| | Engineered | | | HCA202 | |
| | Glycosites | Main N-glycan | | (% of H- | |
| | (Eu | structure of N297 | Main N-glycan structure on | A2F | |
| Antibody | numbering) | (Fc) | engineered glycosites | antibody) | N-Glycan |
| H-A2F | None | 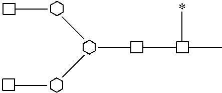 | NA | 100 | 71% A2F |
| A-M3 | None | 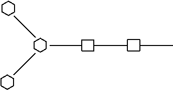 | NA | 87 | 98% M3 |
| A-84- A2G2 | HC: K84N | 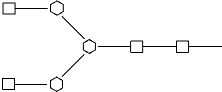 | 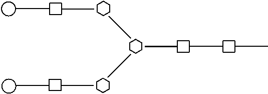 | 89 | Fab: 79% A2G2/ 2% M3 Fc: 63% A2/25% M3 |
| A-8486- A2G2 | LC: D86N HC: K84N | 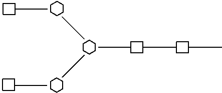 | 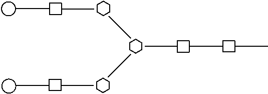 | 82 | Fab: 92% A2G2 Fc: 64% A2 |
| A-8486- A2G2S2 | LC: D86N HC: K84N | 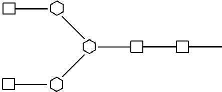 | 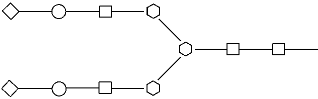 | 108 | Fab: 48% S2/ 27% S1 Fc: 52% A2/ 2% M3 |

NA: not applicable. Main N-glycan structure on Fc is on the canonical N-297 position. Main N-glycan structure on Fab means on the
engineered glycosite by point mutations (Eu numbering). LC is light chain, HC is heavy chain. Binding to HCA202 was performed by ELISA and
compared binding EC₅₀ values. Hexagon represents mannose (Man), white square is N-acetyl glucosamine (GlcNAc), white circle is galactose (Gal), triangle
is N-acetyl galactosamine (GalNAc), white diamond is sialic acid, N-acetyl neuraminic acid (Neu5Ac), and asterisk is fucose (Fuc).

The experimental method is described in Example III.

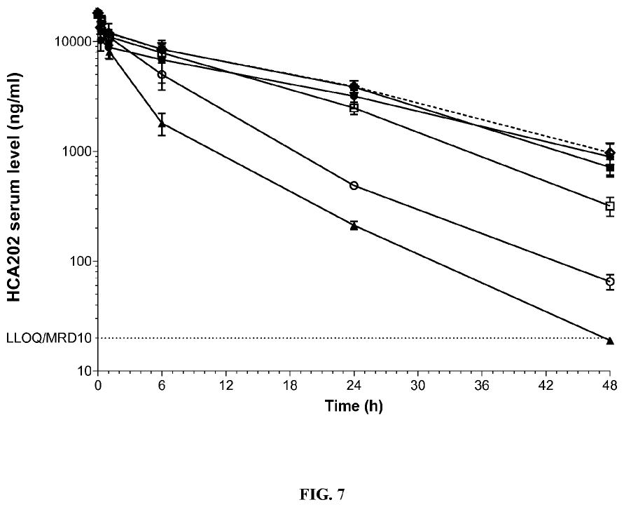

FIG. 7 shows the data obtained for HCA202 levels. When no antibody is injected (PBS condition), HCA202 decays slowly over period of 48 hours, as expected for a Fab fragment. H-A2F (non-engineered adalimumab) treatment led to increase levels of HCA202 at 24 and 48 hours time point. Similarly treatment with A-M3 and A-8486-A2G2S2 led to increased HCA202 levels as compared to PBS treatment (72% depletion from Czero with PBS vs 52-63% depletion with H-A2F, A-M3 and A-8486-A2G2S2 at 6h). Czero is the theoretical concentration (of HCA) in serum that would have been achieved immediately post injection, considering immediate homogeneous whole blood distribution. In contrast injection of A-8486-A2G2 led to a significant decrease of HCA202 levels as compared to H-A2F and PBS after 6 hours (90% depletion). Table 8 shows the HCA202 depletion numbers.

TABLE 8
HCA202 depletion by A2G2 glycosylated Fab antibodies

| | | | | A-8486- | A-84- | A-8486- |
|---|---|---|---|---|---|---|
| | PBS | H-A2F | A-M3 | A2G2S2 | A2G2 | A2G2 |
| % depletion at | 39 | 51 | 34 | 31 | 37 | 56 |
| 1 h | | | | | | |
| % depletion at | 72 | 63 | 53 | 52 | 55 | 90 |
| 6 h | | | | | | |

Table 8 shows the % of HCA202 depletion from Czero.

These data highlight that antibodies displaying A2G2 glycans on their Fab fragment have a high potency to eliminate a circulating antigen from blood circulation in a very short time. These data also show that the glycan load displayed per molecule is critical to enable the depletion potency, as only the A-8486-A2G2 antibody with two engineered glycosites per Fab showed depletion, while A-84-A2G2 antibody with a single glycosite per Fab was non-depleting.

### Example V

### A2GalNAc2 Glycosylated Fab Antibodies Lead to Potent In Vivo Depletion of a Blood Circulating Antigen

To assess the potency of antibodies produced in CGP and displaying GalNAc terminated glycans (A2GalNAc2 structure) to deplete a circulating antigen, an experiment in rat was designed. Rats were injected with an antigen and with antibodies displaying A2GalNAc2 glycan structure on their Fab or control antibodies, specific for the antigen. The level of the circulating antigen in the serum of treated animals was quantified along time to measure the extend of depletion of the antigen from peripheral blood compartment. Table 9 shows the antibody characteristics that were included in the study. All antibodies showed aggregate levels below 5% as assessed by size exclusion HPLC method and higher than 94% purity as assessed by reducing polyacrylamide gel electrophoresis analysis and had an endotoxin level lower than 1 EU/mg (LAL assay). All glycoengineered antibodies conserved high binding to HCA202.

TABLE 9
antibody characteristics.

| | | | | Binding | |
|---|---|---|---|---|---|
| | Engineered | | | to target | |
| | glycosites | Main N-glycan | | (% of | |
| | (Eu) | structure on N297 | Main N-glycan structure on | A2F | |
| Antibody | numbering) | (Fc) | engineered glycosites | antibody) | N-Glycan |
| H-A2F | None | 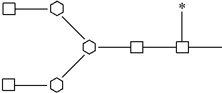 | NA | 100 | 71% A2F |
| A-M3 | None | 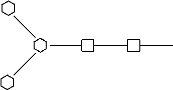 | NA | 87 | 98% M3 |
| A-84- A2GalNAc2 | HC: K84N | 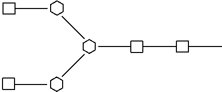 | 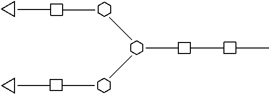 | 87 | Fab: 69% GalNAc2/ 18% GalNAc1 Fc: 92% A2/6% M3 |
| A8486- A2GalNAc2 | LC: D86N HC: K84N | 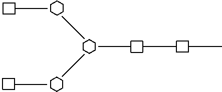 | 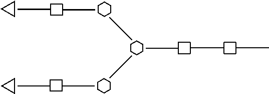 | 88 | Fab: 71% A2GalNAc2/ 15% A2GalNAc1 Fc: 95% A2 |
| A8486- A2G2S2 | LC: D86N HC: K84N | 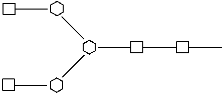 | 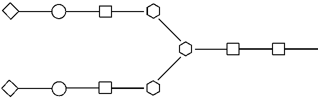 | 108 | Fab: 48% S2/27% S1 Fc: 52% A2/2% M3 |

NA: not applicable. Main N-glycan structure on Fc is on the canonical N-297 position. Main N-glycan structure on Fab means on the
engineered glycosite by point mutations (Eu numbering). LC is light chain, HC is heavy chain. Binding to HCA202 was performed by ELISA and
compared binding EC₅₀ values. Hexagon represents mannose (Man), white square is N-acetyl glucosamine (GlcNAc), white circle is galactose (Gal), triangle
is N-acetyl galactosamine (GalNAc), white diamond is sialic acid, N-acetyl neuraminic acid (Neu5Ac), and asterisk is fucose (Fuc).

The experimental method is described in Example III.

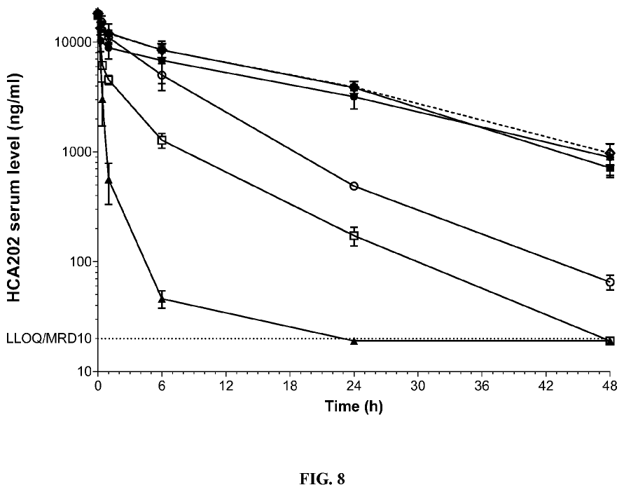

FIG. 8 shows the data obtained for HCA202 levels. When no antibody is injected (PBS condition), HCA202 decays slowly over period of 48h, as expected for a Fab fragment. H-A2F (non-engineered adalimumab) treatment led to increase levels of HCA202 at 24 hours and 48 hours time point. Treatment with A-M3 and A-8486-A2G2S2 also led to increased HCA levels as compared to PBS treatment (72% depletion from Czero with PBS vs 52-63% depletion with H-A2F, A-M3 and A-8486-A2G2S2 at 6h), showing that these antibodies have no depleting potency. Czero is the theoretical concentration (of HCA) in serum that would have been achieved immediately post injection, considering immediate homogeneous whole blood distribution. In contrast injection of A-84-A2GalNAc2 led to a significant depletion of HCA202 as compared to non-depleting antibodies and PBS at 1 hour (74% depletion) and 6 hour (93% depletion). A-8486-A2GalNAc2 treatment led to a more extensive and faster HCA202 depletion (97% depletion at 1 h, 100% at 6h). Table 10 shows the HCA202 depletion numbers.

TABLE 10

| HCA202 depletion by A2GalNAc2 glycosylated Fab antibodies | | | | | | |
|---|---|---|---|---|---|---|
| | | | | A-8486- | A-84- | A-8486- |
| | PBS | H-A2F | A-M3 | A2G2S2 | A2GalNAc2 | A2GalNAc2 |
| % depletion at | 39 | 51 | 34 | 31 | 74 | 97 |
| 1 h | | | | | | |
| % depletion at | | | 53 | 52 | 93 | |
| 6 h | 72 | 63 | | | | 100 |

Table 10 shows the % of HCA202 depletion from Czero.

These data highlight that antibodies displaying A2GalNAc2 glycans on their Fab fragment have a high potency to eliminate a circulating antigen from blood circulation in a very short time. These data also show that the glycan load displayed per molecule modulates the potency of antigen elimination, as the A-8486-A2GalNAc2 antibody with 2 engineered glycosites per Fab showed a higher depletion potency as compared to A-84-A2GalNAc2 antibody with a single glycosite per Fab.

### Example VI

### A2G2 Glycosylated Fab Antibody is Targeted to the Liver In Vivo.

To study the in vivo distribution of antibodies displaying Galactose terminated glycans (A2G2 structure), a study in mouse was designed with fluorescently-labeled antibodies displaying A2G2 or control glycans and using in vivo and ex-vivo tomography imaging.

Antibodies were labeled with CF750 labeling kit (Biotium, ref. 92221) for a volume of at least 1 mL at 1 mg/mL, following manufacturer's instructions. After labeling, the degree of labeling (DOL) was measured. The DOL ranged from 2.7 to 5.2 so antibodies were considered to be similarly labeled. Table 11 shows the antibody characteristics that were included in the study. SKH1 immunocompetent hairless mice (Charles River Laboratories, ref. Crl:SKH1-hr) 5-6 weeks at experiment start were injected (intravenous bolus) with the CF750 labeled antibodies at 5 mg/kg dose. Mice were imaged using the FMT 2500TM fluorescence tomography in vivo imaging in the system (PerkinElmer), which collected both 2D surface fluorescence reflectance images (FRI) as well as 3D fluorescence tomographic (FMT) imaging datasets. For each animal 2 scans in decubitus dorsal position were performed (thorax area+abdomen area) with the FMT (NIR excitation laser 745 nm/emission 770-800 nm) at the following time points: 0.25 h, 1 h, 3h, 6 h, 24 h and 48h. The collected fluorescence data were reconstructed by FMT 2500 system software (TrueQuant V2.0, PerkinElmer) for the quantification of the three-dimensional fluorescence signal in whole animal body. Three-dimensional regions of interest (ROI) were drawn encompassing the relevant biology on the thorax, abdomen and liver areas. The quantity and percentage of dose of labeled antibodies in each ROI was determined for each time point. At time point 6 hour and 48 hour, thyroids (with trachea), lungs, heart, liver, spleen, kidneys were harvested and submitted to FMT imaging

TABLE 11
Antibody characteristics.

| | | Main N-glycan | | |
|---|---|---|---|---|
| | Engineered glycosites | structure on N297 | Main N-glycan structure on | |
| Antibody | (Eu numbering) | (Fc) | engineered glycosites | N-Glycan |
| H-A2F | None | 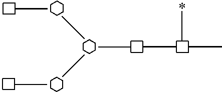 | NA | 71% A2F |
| A-M3 | None | 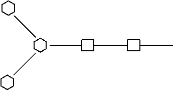 | NA | 98% M3 |
| A-8486- A2G2 | LC: D86N HC: K84N | 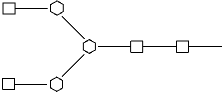 | 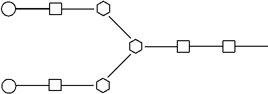 | Fab: 92% A2G2 Fc: 64% A2 |
| A-8486- A2G2S2 | LC: D86N HC: K84N | 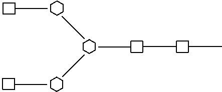 | 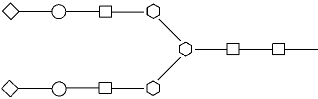 | Fab: 48% S2/ 27% S1 Fc: 52% A2/2% M3 |
| Ptz-A2F | None | 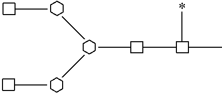 | NA | Fc: 80% A2F |

NA: not applicable. Main N-glycan structure on Fc is on the canonical N-297 position. Main N-glycan structure on Fab means on the engineered glycosite
by point mutations (Eu numbering). LC is light chain, HC is heavy chain. Hexagon represents mannose (Man), white square is N-acetyl glucosamine
(GlcNAc), white circle is galactose (Gal), triangle is N-acetyl galactosamine (GalNAc), white diamond is sialic acid, N-acetyl neuraminic acid (Neu5Ac),
and asterisk is fucose (Fuc).

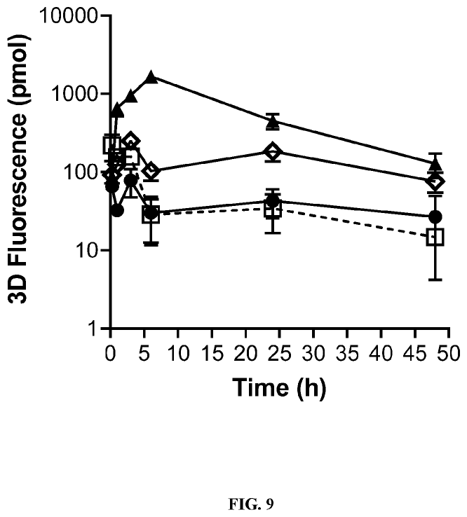

FIG. 9 shows the FMT imaging data for thorax and liver ROI along time for each antibody. Table 12 shows the FMT imaging data obtained on harvested organs at 6 hours. The 2 non engineered control antibodies (H-A2F=adalimumab; Ptz-A2F=pertuzumab) showed a very similar distribution profile with an overall similar and low level of signal distributed in thorax or liver area. Signal did not increase over time in the liver area (FIG. 9). At 6 hour time point, less than 8% of injected dose of Ptz-A2F and H-A2F was present in the liver (Table 12) while these 2 antibodies could be detected in most other organs, albeit at low level. This distribution pattern is consistent with normal human IgG and characteristics of antibodies that are broadly distributed and still mainly present in the blood. The antibodies A-8486-A2G2S2 and A-M3 showed a distribution profile similar to H-A2F and Ptz-A2F control antibodies, with broad organ distribution and relatively low liver distribution. At 6 hour, approximately 20% of the injected dose for A-8486-A2G2S2 and A-M3 was present in the liver. In contrast, A-8486-A2G2 antibody showed a rapid and highly preferential distribution to the liver area (FIG. 9) with a peak at 6 hour time point, followed by a decline after 6 hours. The decline observed after 6 hours is likely due to degradation of the antibody into peptides by the hepatic cells and excretion of the product of degradation (including the fluorophore-couple peptides) outside the liver cells. At 6 hour time point, 75% of the injected dose of A-8486-A2G2 was present in the liver (Table 12). A-8486-A2G2 antibody was absent from Thyroid, lungs, heart and spleen and detectable at low level in the kidneys. This pattern of distribution is characteristics of an antibody that is essentially exclusively distributed to the liver and therefore depleted from the blood. These data show that an antibody displaying A2G2 structure on its Fab fragment is directed almost exclusively to the liver, while capping galactose residues with sialic acid (A-8486-A2G2S2), quenches this effect. These data agree with the observation that A-8486-A2G2 antibody leads to a significant depletion of a circulating antigen in few hours (Example IV). These data support the assumption that A2G2 glycans are recognized by ASGPR on hepatocyte cells, which mediates their internalization and routing to the lysosomal degradation pathway.

TABLE 12
Organ distribution at 6 hour time point

| | | | | A-8486- | A-8486- |
|---|---|---|---|---|---|
| | H-A2F | Ptz-A2F | A-M3 | A2G2S2 | A2G2 |
| Liver | 6.1 | 7.8 | 16.4 | 20.2 | 75.4 |
| Lungs | 1.1 | 2.1 | 2.0 | 1.3 | 0.0 |
| Thyroid/ | 0.5 | 0.9 | 0.4 | 0.1 | 0.0 |
| Trachea | | | | | |
| Heart | 0.7 | 1.3 | 0.8 | 0.9 | 0.0 |
| Kidneys | 2.0 | 3.4 | 2.3 | 1.4 | 1.2 |
| Spleen | 0.3 | 0.2 | 0.7 | 0.5 | 0.0 |

Table 12 shows the average (N = 3) % of injected fluorophore dose in indicated harvested organs at 6 hour time point.

### Example VII

### A2GalNAc2 Glycosylated Fab Antibody is Targeted to the Liver In Vivo.

To study the in vivo distribution of antibodies displaying GalNAc terminated glycans (A2GalNAc2 structure), a study in mouse was designed with fluorescently-labeled antibodies displaying A2GalNAc2 or control glycans and using in vivo and ex-vivo tomography imaging. The protocol and study outline are described in Example VI. Table 13 shows the antibody characteristics that were included in the study.

TABLE 13
Antibody characteristics.

| | | Main N-glycan | | |
|---|---|---|---|---|
| | Engineered glycosites | structure on N297 | Main N-glycan structure on | |
| Antibody | (Eu numbering) | (Fc) | engineered glycosites | N-Glycan |
| H-A2F | None | 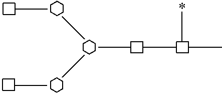 | NA | 71% A2F |
| A-M3 | None | 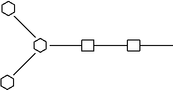 | NA | 98% M3 |
| A-8486- A2GalNAc2 | LC: D86N HC: K84N | 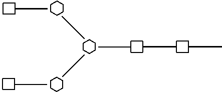 | 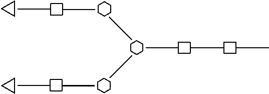 | Fab: 71% A2GalNAc2/ 15% A2GalNAc1 Fc: 95% A2 |
| A-8486- A2G2S2 | LC: D86N HC: K84N | 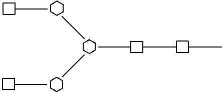 | 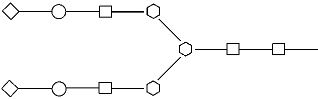 | Fab: 48% S2/ 27% S1 Fc: 52% A2/ 2% M3 |
| Ptz-A2F | None | 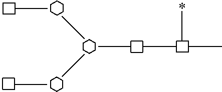 | NA | Fc: 80% A2F |

NA: not applicable. Main N-glycan structure on Fc is on the canonical N-297 position. Main N-glycan structure on Fab means on the engineered glycosite
by point mutations (Eu numbering). LC is light chain, HC is heavy chain. Hexagon represents mannose (Man), white square is N-acetyl glucosamine
(GlcNAc), white circle is galactose (Gal), triangle is N-acetyl galactosamine (GalNAc), white diamond is sialic acid, N-acetyl neuraminic acid (Neu5Ac),
and asterisk is fucose (Fuc).

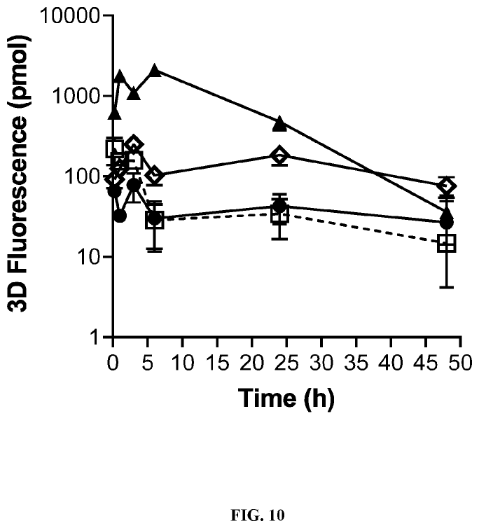

FIG. 10 shows the FMT imaging data for thorax and liver ROI along time for each antibody. Table 14 shows the FMT imaging data obtained on harvested organs at 6 hours. The 2 non-engineered control antibodies (H-A2F=adalimumab; Ptz-A2F=pertuzumab) showed a very similar distribution profile with an overall similar and low level of signal distributed in thorax or liver area. Signal did not increase over time in the liver area (FIG. 10). At 6 hour time point, less than 8% of injected dose of Ptz-A2F and H-A2F was present in the liver (Table 14) while these 2 antibodies could be detected in most other organs. This distribution pattern is consistent with normal human IgG and characteristics of antibodies that are broadly distributed and still mainly present in the blood. The antibodies A-8486-A2G2S2 and A-M3 showed a distribution profile similar to H-A2F and Ptz-A2F control antibodies, with broad organ distribution and relatively low liver distribution. At 6 hour, 20% of the injected dose for A-8486-A2G2S2 and A-M3 was present in the liver (Table 14). In contrast, A-8486-A2GalNAc2 antibody showed a rapid and essentially exclusive distribution to the liver area, followed by a decline after 6 hours (FIG. 10). The decline observed after 6 hours is likely due to degradation of the antibody into peptides by the lysosomal machinery and excretion of the product of degradation (including the fluorophore-couple peptides) from the liver cells. At 6 hour time point, 85% of the injected dose of A-8486-A2GalNAc2 was present in the liver (Table 14). A-8486-A2GalNAc2 antibody was absent from Thyroid, lungs, heart and spleen and detectable at low level in the kidneys. This pattern of distribution is characteristics of an antibody that is essentially exclusively distributed to the liver and therefore not present in the blood and other organs.

These data show that an antibody displaying A2GalNAc2 structure on its Fab fragment is directed almost exclusively to the liver. These data agree with the observations that GalNAc2 Fab antibodies are specifically internalized via ASGPR by HepG2 hepatocyte cells (Example II) and that GalNAc2 Fab antibodies mediate a potent and fast depletion of a circulating antigen (Example VII) from peripheral blood. Altogether, these data indicate that A2GalNAc2 glycans displayed on Fab fragment of antibodies are efficiently recognized by ASGPR on hepatocyte cells, which mediates their internalization and routing to the lysosomal degradation pathway.

TABLE 14
Organ distribution data at 6 hour.

| | | | | A-8486- | A-8486- |
|---|---|---|---|---|---|
| | H-A2F | Ptz-A2F | A-M3 | A2G2S2 | A2GalNAc2 |
| Liver | 6.1 | 7.8 | 16.4 | 20.2 | 85.3 |
| Lungs | 1.1 | 2.1 | 2.0 | 1.3 | 0.0 |
| Thyroid/ | 0.5 | 0.9 | 0.4 | 0.1 | 0.0 |
| Trachea | | | | | |
| Heart | 0.7 | 1.3 | 0.8 | 0.9 | 0.0 |
| Kidneys | 2.0 | 3.4 | 2.3 | 1.4 | 1.3 |
| Spleen | 0.3 | 0.2 | 0.7 | 0.5 | 0.1 |

Table 14 shows the average (N = 3) % of injected fluorophore dose in indicated harvested organs at 6 hour time point.

### Example VIII

### A2 Glycosylated Fab Antibody is Targeted to the Liver In Vivo.

To study the in vivo distribution of antibodies displaying GlcNAc terminated glycans (A2 structure), a study in mouse was designed with fluorescently-labeled antibodies displaying A2 or control glycans and using in vivo and ex-vivo tomography imaging. The protocol and study outline is described in Example VI. Table 15 shows the antibody characteristics that were included in the study.

TABLE 15
Antibody characteristics.

| | | Main N-glycan | | |
|---|---|---|---|---|
| | Engineered glycosites | structure on N297 | Main N-glycan structure on | |
| Antibody | (Eu numbering) | (Fc) | engineered glycosites | N-Glycan |
| H-A2F | None | 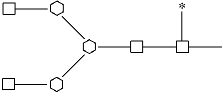 | NA | 71% A2F |
| A-M3 | None | 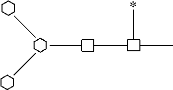 | NA | 98% M3 |
| A-8486-A2 | LC: D86N HC: K84N | 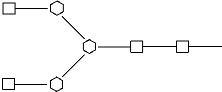 | 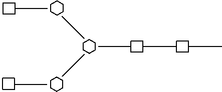 | Fab: 98% A2 Fc: 96% A2 |
| A-8486- A2G2S2 | LC: D86N HC: K84N | 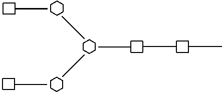 | 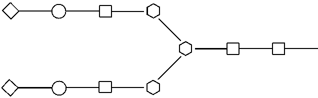 | Fab: 48% S2/ 27% S1 Fc: 52% A2/2% M3 |
| Ptz-A2F | None | 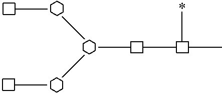 | NA | Fc: 80% A2F |

NA: not applicable. Main N-glycan structure on Fc is on the canonical N-297 position. Main N-glycan structure on Fab means on the engineered glycosite
by point mutations (Eu numbering). LC is light chain, HC is heavy chain. Hexagon represents mannose (Man), white square is N-acetyl glucosamine
(GlcNAc), white circle is galactose (Gal), triangle is N-acetyl galactosamine (GalNAc), white diamond is sialic acid, N-acetyl neuraminic acid (Neu5Ac),
and asterisk is fucose (Fuc).

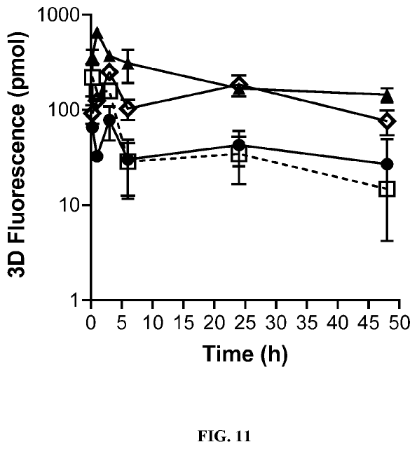

FIG. 11 shows the FMT imaging data for thorax and liver ROI along time for each antibody. Table 16 shows the FMT imaging data obtained on harvested organs at 6 hours. The two non engineered control antibodies (H-A2F=adalimumab; Ptz-A2F=pertuzumab) showed a very similar distribution profile with an overall similar and low level of signal distributed in thorax or liver area. Signal did not increase over time in the liver area (FIG. 11). At 6 hour time point, less than 8% of injected dose of Ptz-A2F and H-A2F was present in the liver (Table 16) while these 2 antibodies could be detected in most other organs. This distribution pattern is consistent with normal human IgG and characteristics of antibodies that are broadly distributed and still mainly present in the blood. The antibodies A-8486-A2G2S2 and A-M3 showed a distribution profile similar to H-A2F and Ptz-A2F control antibodies, with broad organ distribution and relatively low liver distribution. A-8486-A2 antibody showed a rapid and preferential distribution to the liver area (peak at 1h), followed by a rapid decrease at 6 hour and slow decrease between 6 hour and 48 hours (FIG. 11). At 6 hour time point, 20% of the injected dose of A-8486-A2 was present in the liver (Table 16). A-8486-A2 antibody was absent from Thyroid, lungs, heart and kidneys and detectable at low level in the spleen. This pattern of distribution is characteristic of an antibody that is not present in the blood. This is consistent with the data showing that A-8486-A2 led to a fast and potent depletion of a circulating antigen (Example III). These data support the assumption that antibodies displaying A2 structure on their Fab fragment are recognized by a specific glycan receptor, which triggers internalization and routing to the lysosomal degradation pathway.

TABLE 16
Organ distribution data at 6 hour.

| | H-A2F | Ptz-A2F | A-M3 | A-8486-A2G2S2 | A-8486-A2 |
|---|---|---|---|---|---|
| Liver | 6.1 | 7.8 | 16.4 | 20.2 | 19.6 |
| Lungs | 1.1 | 2.1 | 2.0 | 1.3 | 0.0 |
| Thyroid/ | 0.5 | 0.9 | 0.4 | 0.1 | 0.0 |
| Trachea | | | | | |
| Heart | 0.7 | 1.3 | 0.8 | 0.9 | 0.0 |
| Kidneys | 2.0 | 3.4 | 2.3 | 1.4 | 0.0 |
| Spleen | 0.3 | 0.2 | 0.7 | 0.5 | 2.4 |

Table 16 shows the average (N = 3) % of injected fluorophore dose in indicated harvested organs at 6 hour time point.

### Example IX

### A2GalNAc2 Glycosylated Antibody Leads to Target Receptor Degradation on Surface of ASGPR Expressing Cells.

To assess whether an antibody specific for a target receptor, and displaying GalNAc2 terminated glycan (A2GalNAC2 structure) can lead to degradation of the target surface receptor on surface of cells expressing ASGPR, an experiment using CGP-produced glycovariants of the pertuzumab (Ptz) anti-HER2 antibody was performed. HepG2 cells co-express HER2 and ASGPR. The hypothesis tested was that A2GalNAc2 displaying Ptz CGP-produced variant is able to co-engage HER2 and ASGPR on surface of HepG2 cells and trigger internalization and degradation of the formed complex, leading to reduction of HER2 levels on HepG2 cells. Pertuzumab antibodies (Ptz-A2, Ptz-86-A2GalNAc2, Ptz-gt1-A2GalNAc2, Ptz-hgt-A2GalNAc2) were purified from cell culture supernatant with Protein A HiTrap Mabselect PrismA, (Cytiva) and formulated in PBS buffer pH 7, by using Amicon Concentrator, (4 ml, 30K MWCO). Due to lower levels of A2GalNAc2 (<70%) on the purified antibodies, the material was further polished by in-vitro glycoengineering to increase abundance of A2GalNAc2 glycan on Ptz. The GalNAc addition was performed using in vitro glycosylation (IVGE) in a reaction using 10 mM UDP-GalNAc, 2% (w/w) GalT1 (Y285L), 100 mM MnCl2 in 25 mM Tris, pH 8 at 30° C. under mild rotation. The glycosylated mAb was purified from the reaction mixture with ProteinA sepharose (HiTrap MabSelect PrismA column GE Healthcare) according to manufacturer's recommendation using FPLC (Bio-Rad NGC, Germany). Thereafter, a desalting procedure using PD-10 (Sephadex 25, Sigma, Switzerland) was carried out for a buffer exchange to PBS pH 7. Sample was then treated with a Pierce High-Capacity Endotoxin Removal Spin Column, 0.25 mL (cat: 88273, Thermo), sterile filtrated, and diluted to 0.05 mg/ml with PBS pH 7. The resulting antibodies and their glycan structure used in this experiment are described in Table 17. Ptz-gt1 antibody comprises an inserted glycotag at the C-terminal part of the heavy chain (ANSTMMS addition with C-terminal lysine replaced by Alanine of the glycotag sequence). Ptz-hgt antibody comprises an inserted glycosite in the upper hinge region (LNLSS insertion after T223 position).

TABLE 17
Antibody characteristics.

| | | | | Binding | | Endo- |
|---|---|---|---|---|---|---|
| | | | | to Her2 | | toxin |
| | Engineered | Main N-glycan | | (% of | | (EU/mg). |
| | glycosites | structure of N297 | Main N-glycan structure on | Ptz-A2F | | Assay |
| Antibody | (Eu numbering) | (Fc) | engineered glycosites | antibody) | N-Glycan | LAL. |
| Ptz-A2F | None | 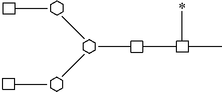 | NA | 100 | Fc: 80% A2F | <0.5 EU/mg |
| Ptz-A2 | None | 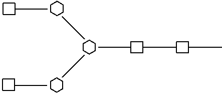 | NA | 74 | Fc: 97% A2 | <1.0 Eu/mg |
| Ptz-86- A2GalNAc2 | LC: D86N | 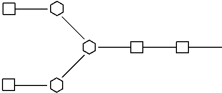 | 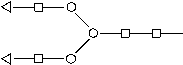 | 85 | Fc: 24% A2GalNAc2/ 52% A2 Fab: 76% A2GalNAc2 | <1.0 EU/mg |
| Ptz-gt1- A2GalNAc2 | HC C- terminal K was replaced by A with addition of NSTMMS* | 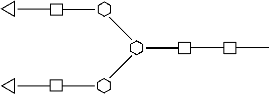 | 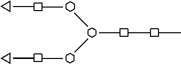 | 51 | Total: 72% A2GalNAc2 | <1.0 EU/mg |
| Ptz-hgt- A2GalNAc2 | HC Insertion of LNLSS after T223 at upper hinge | 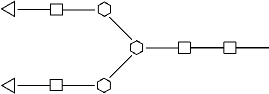 | 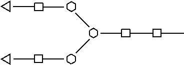 | 76 | Fab: 94% A2GalNAc2 Fc: 60.7% A2GalNAc2 | <0.5 EU/mg |

NA: not applicable. Main N-glycan structure on Fc is on the canonical N-297 position. Main N-glycan structure on Fab means on the engineered glycosite
by point mutations (Eu numbering). LC is light chain, HC is heavy chain. LAL: limulus amebocyte lysate. Binding to Her2 was done by ELISA at 200
ng/ml. Hexagon represents mannose (Man), white square is N-acetyl glucosamine (GlcNAc), white circle is galactose (Gal), triangle is N-acetyl
galactosamine (GalNAc), white diamond is sialic acid, N-acetyl neuraminic acid (Neu5Ac), and asterisk is fucose (Fuc).

hepatocarcinoma cells (ATCC #HB-8065) were maintained in a low glucose DMEM medium (Sigma, Ref. D5546) supplemented with 10% FBS and 2 mM Glutamine. HepG2 cells were harvested using Accutase (Sigma/Merck, SCR005) and plated in flat-bottom 24-well plates at 0.1 million cells/well. Cells were left to recover for 72 hours at 37° C. in a cell culture incubator. Cells were then incubated for 24 hours with non-engineered pertuzumab (Ptz-A2F; Perjeta, obtained from pharmacy) or CGP-produced pertuzumab glycovariant antibodies at 1 μg/ml+IVIg (Hizentra, obtained from pharmacy) at 1 mg/ml, in cell culture medium, at 37° C. in cell culture incubator. Cells were then harvested and stained with anti-HER2 antibody (Human ErbB2/HER2 antibody, R&D, Ref. MAB1129) at 1 μg/ml and anti-mouse IgG-fluorochrome secondary antibody (Goat anti-Mouse IgG (H+L) Cross-Adsorbed Secondary Antibody (PE), ThermoFisher, P-852), used at 2 μg/ml, according to standard flow cytometry staining protocol. Antibody MAB1129 was shown to be non-competitive with pertuzumab for binding to HER2, ensuring that MAB1129 can bind to HER2 even if pertuzumab is bound. Cells were immediately acquired on a flow cytometer and analyzed using FlowJo (TriStar) software. The geometric mean fluorescence intensity (MFI) of HER2 was extracted for each condition. The HER2 MFI were adjusted to the pHrodo DOL (adjusted MFI). The adjusted MFI values were expressed as % of Ptz-A2F (normalized MFI).

Table 18 presents the adjusted and normalized HER2 MFI. Treatment with the control antibody Ptz-A2, which has no engineered glycosite and displays an A2 structure on the N297 Fc site did not reduce HER2 levels as compared to Ptz-A2F (107% of normalized HER2 MFI after treatment). In contrast, treatment with Ptz-gt1-A2GalNAc2 reduced normalized HER2 MFI to 64% and treatment with Ptz-hgt-A2GalNAc2 reduced normalized HER2 MFI to 72%. The reduction of HER2 by Ptz-gt1-A2GalNAc2 and Ptz-hgt-A2GalNAc2 was statistically significant compared to Ptz-A2F group (ratio paired t-test on adjusted MFI data) (Table 18). Interestingly, treatment with Ptz-86-A2GalNAc2 did not trigger HER2 degradation as compared to Ptz-A2F (95% normalized HER2 MFI). All the antibodies showed a similar HER2 binding capacity (Table 17), ruling out that different HER2 degradation capacity is linked to a reduced HER2 binding potency. Moreover A2GalNAc2 antibodies containing the equivalent engineered glycosite position 86 in the Fab fragment did show efficient uptake by HepG2 cells (Example II), i and high depletion potency of a circulating antigen, when injected in animals indicating efficient ASGPR engagement (Example V), indicating that position 86 displays accessible, active glycans. This indicates that the position of glycan displayed on the antibody is important to enable efficient engagement of ASGPR when the antibody is bound on HER2. Altogether this study shows that A2GalNAc2 displaying antibodies can be used to remove a target molecule from a cell surface, by leveraging the ASGPR endocytic and lysosomal degradation pathway.

TABLE 18
HER2 degradation data on HepG2

| | | | Ptz-86- | Ptz-hgt- | Ptz-gt1- |
|---|---|---|---|---|---|
| | Ptz-A2F | Ptz-A2 | A2GalNAc2 | A2GalNAc2 | A2GalNAc2 |
| Mean | 100.0 (±0.0) | 107.1 (±3.6) | 95.30 (±3.8) | 71.83 (±3.2) | 64.13 (±3.5) |
| normalized | | | | | |
| HER2 MFI | | | | | |
| (±SEM) | | | | | |
| 95% CI of the | 100-100 | 91.75-122.5 | 78.75-111.9 | 58.04-85.62 | 49.19-79.08 |
| Mean | | | | | |
| normalized | | | | | |
| Mean adjusted | 3773 (±1061) | 4082 (±1206) | 3613 (±1037) | 2706 (±772.2) | 2396 (±649.5) |
| MFI (±SEM) | | | | | |
| p value vs Ptz- | | p > 0.05 | p > 0.05 | p = 0.017 | p = 0.014 |
| A2F | | | | | |

SEM: standard error of the mean. CI: confidence interval. P value was calculated with a ratio paired t-test on adjusted MFI data.

### REFERENCES

[1] Pokhrel et al. “Graves disease.” StatPearls [Internet] (2020).
[2] Lane et al. “New therapeutic horizons for Graves' hyperthyroidism.” Endocrine Reviews 41.6 (2020): 873-884.
[3] O'Connor et al. “CCL2-CCR2 signaling in disease pathogenesis.” Endocrine, Metabolic & Immune Disorders-Drug Targets (Formerly Current Drug Targets-Immune, Endocrine & Metabolic Disorders) 15.2 (2015): 105-118.
[4] Gschwandtner et al. “More than just attractive: how CCL2 influences myeloid cell behavior beyond chemotaxis.” Frontiers in immunology 10 (2019): 2759.
[5] Liu et al. “Sox9 regulates self-renewal and tumorigenicity by promoting symmetrical cell division of cancer stem cells in hepatocellular carcinoma.” Hepatology 64.1 (2016): 117-129. [6] Jo et al. Cross-talk between epidermal growth factor receptor and c-Met signal pathways in transformed cells. J Biol Chem 275 (2000): 8806-8011.
[7] Kim et al. “Targeting wnt signaling for gastrointestinal cancer therapy: Present and evolving views.” Cancers 12.12 (2020): 3638.
[8] Ciardiello et al. “Clinical development of therapies targeting TGFβ: current knowledge and future perspectives.” Annals of Oncology (2020).
[9] Zhang et al. “Tumor-associated macrophages: A promising target for a cancer immunotherapeutic strategy.” Pharmacological Research 161 (2020): 105111.
[10] van Die et al. “Essentials of Glycobiology.” Essentials of Glycobiology. 3rd edition. (2017).
[11] Braun, et al. “The major subunit of the asialoglycoprotein receptor is expressed on the hepatocellular surface in mice lacking the minor receptor subunit.” Journal of Biological Chemistry 271.35 (1996): 21160-21166.
[12] Ippoliti et al. “The effect of monensin and chloroquine on the endocytosis and toxicity of chimeric toxins.” Cellular and Molecular Life Sciences CMLS 54.8 (1998): 866-875.

1. A glycoengineered bifunctional binding protein comprising: a first moiety that specifically binds to a target protein associated with a disease; and at least one second moiety that binds specifically to an endocytic carbohydrate-binding protein or receptor, wherein the at least one second moiety comprises a glycan structure.

2. The glycoengineered bifunctional binding protein of claim 1, wherein the at least one second moiety comprises glycan comprising terminal GlcNAc.

3. The glycoengineered bifunctional binding protein of claim 1, wherein the at least one second moiety comprises a glycan comprising terminal GalNAc.

4. The glycoengineered bifunctional binding protein of claim 1, wherein the at least one second moiety comprises a glycan comprising terminal Gal.

5. The glycoengineered bifunctional binding protein of claim 1, wherein the at least one second moiety comprises a glycan comprising the structure: 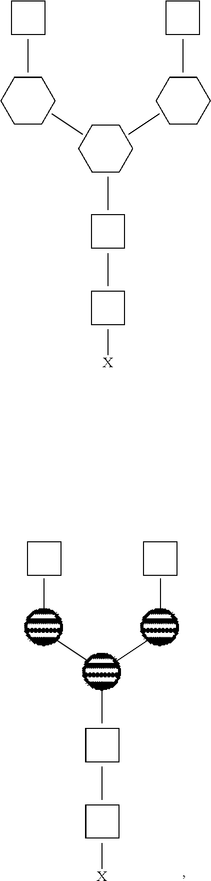
wherein the square represents an N-acetylglucosamine (GlcNAc) residue and the hexagon represents a mannose (Man) residue, and wherein X represents an amino acid residue of the bifunctional binding protein.

6. The glycoengineered bifunctional binding protein of claim 1, wherein the at least one second moiety comprises a glycan comprising the structure: 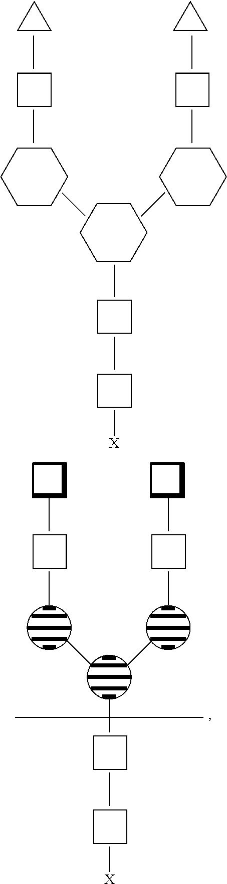
wherein the triangle represents an N-acetyl galactosamine (GalNAc), the white square represents an N-acetylglucosamine (GlcNAc) residue and the hexagon represents a mannose (Man) residue, and wherein X represents an amino acid residue of the bifunctional binding protein.

7. The glycoengineered bifunctional binding protein of claim 1, wherein the at least one second moiety comprises a glycan comprising the structure: 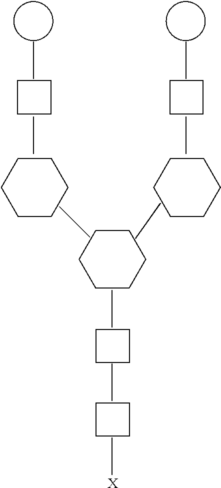
wherein the white circle represents a galactose (Gal) residue, the square represents an N-acetylglucosamine (GlcNAc) residue and the hexagon represents a mannose (Man) residue, and wherein X represents an amino acid residue of the bifunctional binding protein.

8. The glycoengineered bifunctional binding protein of claim 1, wherein the bifunctional binding protein (i) specifically binds to a target protein and (ii) comprises at least one N-glycan of the structure: 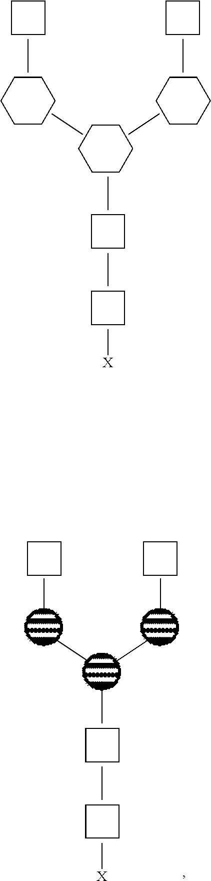
wherein the at least one N-glycan is linked to the bifunctional binding protein at 1, 2, 3, 4 or 5 N-glycosylation sites.

9. (canceled)

10. (canceled)

11. (canceled)

12. The glycoengineered bifunctional binding protein of claim 1, wherein the bifunctional binding protein (i) specifically binds to a target protein and (ii) comprises at least one N-glycan of the structure: 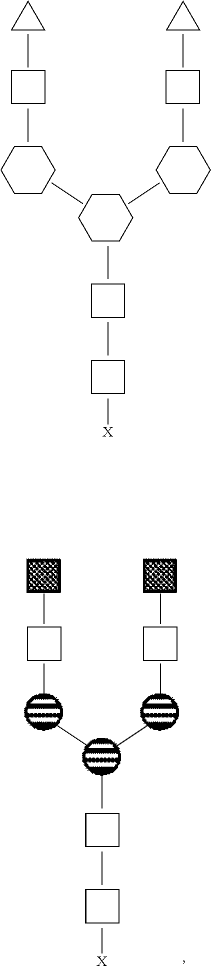
wherein the at least one N-glycan is linked to the bifunctional binding protein at 1, 2, 3, 4 or 5 N-glycosylation sites.

13. The glycoengineered bifunctional binding protein of claim 12, wherein any branched structure of the N-glycan of the proximal GlcNAc can be included.

14. The glycoengineered bifunctional binding protein of claim 12, wherein a GlcNAc residue of the N-glycan is fucosylated.

15. (canceled)

16. The glycoengineered bifunctional binding protein of claim 1, wherein the bifunctional binding protein (i) specifically binds to a target protein and (ii) comprises at least one N-glycan of the structure: 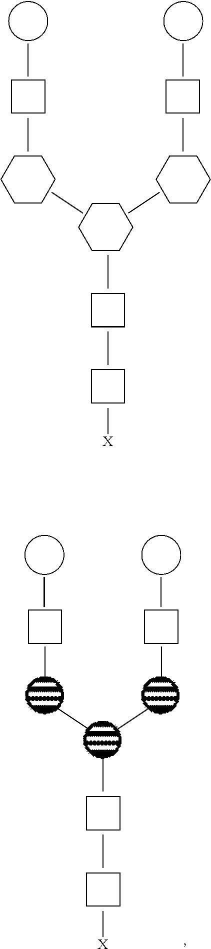
wherein the at least one N-glycan is linked to the bifunctional binding protein at 1, 2, 3, 4 or 5 N-glycosylation sites.

17. (canceled)

18. (canceled)

19. (canceled)

20. The glycoengineered bifunctional binding protein of claim 6, wherein the amino acid residue is Asn.

21. (canceled)

22. The glycoengineered bifunctional binding protein of claim 1, wherein the glycan structure comprises GlcNAc2Man3GlcNAc2, GalNAc2GlcNAc2Man3GlcNAc2, Gal2GlcNAc2Man3GlcNAc2, GlcNAc1Man3GlcNAc2, Gal2GlcNAc2Man3GlcNAc2, Gal1GlcNAc2Man3GlcNAc2, GalNAc1GlcNAc2Man3GlcNAc2, GlcNAc3Man3GlcNAc2, GlcNAc4Man3GlcNAc2, Gal3GlcNAc3Man3GlcNAc2, GalNAc3GlcNAc3Man3GlcNAc2, GalNAc4GlcNAc4Man3GlcNAc2, Gal4GlcNAc4Man3GlcNAc2, or Man-6-P-N-glycan.

23. (canceled)

24. (canceled)

25. (canceled)

26. The glycoengineered bifunctional binding protein of claim 1, wherein the bifunctional protein is an antibody or fragment thereof and comprises (a) a heavy chain variable region; (b) a light chain variable region; or (c) an Fab fragment.

27. (canceled)

28. (canceled)

29. (canceled)

30. (canceled)

31. (canceled)

32. The glycoengineered bifunctional binding protein of claim 26, wherein the antibody or fragment thereof has a glycan to protein ratio of at least 2 to 1, at least 4 to 1, at least 6 to 1, at least 8 to 1, or at least 10 to 1.

33. (canceled)

34. The glycoengineered bifunctional binding protein of claim 1, wherein the glycoengineered bifunctional binding protein binds to an autoantibody and comprises an autoantigen or immunogenic fragment thereof.

35. A population of the glycoengineered bifunctional binding protein of claim 1, wherein at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% of the glycans at a given glycosite are the same.

36. (canceled)

37. (canceled)

38. (canceled)

39. (canceled)

40. (canceled)

41. The glycoengineered bifunctional binding protein of claim 1, wherein the target protein associated with a disease is: (i) upregulated in the disease compared to a non-disease state; (ii) expressed in the disease compared to a non-disease state; or (iii) involved in cancer progression.

42. (canceled)

43. (canceled)

44. The glycoengineered bifunctional binding protein of claim 1, wherein the target protein is TNFα, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPα, CD38, CD73, or TGF-β, Bombesin R, CAIX, CD13, CD44, v6, Emmprin, Endoglin, EpCAM, EphA2, FAP-α, Folate R, GRP78, IGF-1R, Matriptase, Mesothelin, sMET/HGFR, MT1-MMP, MT6-MMP, PSCA, PSMA, Tn antigen, and uPAR, TSHRα, MOG, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, LILRB1, LILRB2, VEGF-R, CXCR4, CXCL12, CSF-1, CD47, aggregated light chains or aggregated transthyretin.

45. (canceled)

46. The glycoengineered bifunctional binding protein of claim 1, wherein the endocytic carbohydrate-binding protein or receptor is a DC-SIGN, L-SIGN, LSECTin, asialoglycoprotein receptor (ASGPR), mannose-6-phosphate receptor, mincle, dectin-1, dectin-2, langerin, cation-independent mannose 6-phosphate receptor (CI-MPR), macrophage mannose receptor 2, BDCA-2, MGL, MDL, MICL, CLEC2, DNGR1, or CLEC12B.

47. (canceled)

48. (canceled)

49. A method of delivering a target protein to a hepatocyte endosome comprising: contacting the target protein with the glycoengineered bifunctional binding protein of claim 1 under conditions sufficient to mediate endocytosis of the target protein.

50. (canceled)

51. (canceled)

52. (canceled)

53. (canceled)

54. (canceled)

55. (canceled)

56. A method of degrading a target protein comprising: contacting the target protein with the glycoengineered bifunctional binding protein of claim 1 under conditions sufficient to mediate lysosomal degradation of the target protein by a host cell.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. A pharmaceutical composition comprising the glycoengineered bifunctional binding protein of claim 1 and a pharmaceutically acceptable carrier.

76. A method of treating or preventing a disease in a patient comprising: administering to the patient the glycoengineered bifunctional binding protein of claim 1.

77. (canceled)

78. (canceled)

79. (canceled)

80. (canceled)

81. (canceled)

82. (canceled)

83. (canceled)

84. A kit comprising the glycoengineered bifunctional binding protein of claim 1 and instructions for administering the glycoengineered bifunctional molecule to an individual in need thereof.

85. (canceled)

86. (canceled)

87. A method of degrading a target protein in a subject comprising administering a bifunctional binding protein, wherein the bifunctional binding protein specifically binds to the target protein and comprises a biantennary GalNAc capable of binding asialoglycoprotein receptor (ASGPR).

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)
